(19) United States
(12) Patent Application Publication
(10) Pub. No.: US 2003/0113762 A1
(43) Pub. Date: Jun. 19, 2003

(54) Gleason grade 4/5 prostate cancer genes

(76) Inventor: Janet A. Warrington, Los Altos, CA (US)

Correspondence Address: Alison B. Mohr, Parsons Behle & Latimer, Suite 1800, 201 South Main Street, Salt Lake City, UT, 84111-2218, US

(21) Appl. No.: 10/222,206

(22) Filed: Aug. 16, 2002

Related U.S. Application Data
(60) Provisional application No. 60/312,745, filed on Aug. 17, 2001.

(51) Int. Cl. C12Q 1/68; G01N 33/574; G06F 19/00; G01N 33/48; G01N 33/50

(52) U.S. Cl. 435/6; 435/7.23; 702/19; 702/20

(57) ABSTRACT

Sixty-four down regulated and 22 up regulated genes have been indentified in Gleason grade 4/5 cancer, using the gene profile from benign prostatic hyperplasia as control tissue. Hepsin appears to be the most promising of the up regulated genes. PSMA is also highly overexpressed at the transcript level in grade 4/5 cancer, re-emphasizing its potential importance as a target for chemotherapy. The regulated genes can be used diagnostically, prognostically, and therapeutically. They can be used to form prostate specific expression monitoring tools.

### RELATED APPLICATIONS

This application claims priority to Provisional Application Serial No. 60/312,745 filed Aug. 17, 2001, which is herein incorporated by reference in its entirety for all purposes.

### FIELD OF THE INVENTION

The invention relates to the field of cancer diagnostics and therapeutics. In particular it relates to prostate cancer.

### BACKGROUND OF THE INVENTION

Prostate cancer, along with lung and colon cancer, are the three most common causes of death from cancer in men in the United States. Greenlee, R. T., Hill-Hannon, M. B., Murray, T., Thun, M., Cancer Statistics, 2001, CA Cancer J Clin, 15, 2001, which is herein incorporated by reference in its entirety. However, prostate cancer is by far the most prevalent of all human malignancies with the exception of skin cancer. Scott, R., Mutchnik, D. L., Laskowski, T. Z., Schmalhorst, W. R., Carcinoma of the prostate in elderly men: Incidence, growth characteristics and clinical significance, J Urol, 101: 602-607,1969 and Sakr, W. A., Haas, G. P., Cassin, B. F., Pontes, J. E., Crissman, J. D., The frequency of carcinoma and intraepithelial neoplasia of the prostate in young male patients, J Urol, 150: 379-385,1993, which are herein incorporated by reference in their entirety.

In previous studies, nine histologic variables related to prostate cancer progression in 379 men with long-term follow-ups after radical prostatectomy were measured using a detectable, rising prostate-specific antigen (PSA) as an indicator of progressive cancer. Stamey, T. A., McNeal, J. E., Yemoto, C. M., Sigal, B. M., Johnstone, I. M., Biological determinants of cancer progression in men with prostate cancer, JAMA, 281: 1395-400,1999, which is herein incorporated by reference in its entirety. It was found that the strongest histologic predictor of progression in radical prostatectomy specimens examined at 3-mm section intervals was the amount of Gleason grade 4/5 tumor in the largest peripheral zone (PZ) cancer. For every 10% increase in Gleason grade 4/5, a proportional 10% increase in post-radical prostatectomy PSA failure rates was found.

Although serum PSA between 2-10 ng/ml has been widely used in the United States as a potential marker for prostate cancer, in this range it is largely related to benign prostatic hyperplasia (BPH), a much more common disease. Roehrborn, C. G., McConnell, J., Bonilla J. et al., Serum prostate specific antigen is a strong predictor of future prostate growth in men with benign prostatic hyperplasia, J Urol, 163: 13, 2000, which is herein incorporated by reference in its entirety. Moreover, it is now know that serum PSA is poorly correlated with the volume of both high-grade (Gleason grade 4/5) and low-grade (Gleason grades 3, 2, and 1) prostate cancer, and that the level of pre-radical prostatectomy PSA does not discriminate between potential cure rates at PSA levels around 2-12 ng/ml. Stamey, T. A., Johnstone, I. M., McNeal, J. E., Preoperative serum PSA levels between 2 and 9 ng/ml correlate poorly with post-radical prostatectomy cancer morphology and PSA cure rates, Submitted to J Urol, May 1, 2001, which is herein incorporated by reference in its entirety. Adding to the PSA dilemma is our recent observation that preoperative positive prostatic biopsies have no dependable relationship to the important characteristics of the largest tumor within the prostate that determines cancer progression. Noguchi, M., Stamey, T. A., McNeal, J. E. et al., Relationship between systematic biopsies and histologic features in 222 radical prostatectomy specimens: Lack of prediction of tumor significance in men with nonpalpable prostate cancer, J Urol, July 2001, which is herein incorporated by reference in its entirety.

There is a need in the art for tumor markers for prostate cancer that can provide alternative measures to the notoriously inaccurate PSA. In particular, there is a need for markers for Gleason grade 4/5 prostate cancer, which is strongly related to poor outcome.

### SUMMARY OF THE INVENTION

According to one aspect of the invention a method is provided for predicting the outcome of cancer in a patient. The level of expression of at least one RNA transcript or its translation product in a first or a second group of RNA transcripts in a first sample of prostate tissue is compared to the level of expression of the transcripts or translation products in a second sample of prostate tissue. The first prostate tissue sample is neoplastic and the second prostate tissue sample is nonmalignant human prostate tissue. The first group of RNA transcripts consists of transcripts of genes selected from the group consisting of genes ranked 1-5,7-8,10-13, 15-17,19-20, 22, 24-25, 27-28, 31, 34-36, 38, 39, 40-43, 45-61, and 62 as shown in Table 4 and the second group of RNA transcripts consists of transcripts of genes selected from the group consisting of genes ranked 1, 3, 5-21, and 22 as shown in Table 3. The patient is identified as having a poor outcome when expression of at least one of the first group of RNA transcripts or translation products is found to be lower in the first sample than in the second sample, or expression of at least one of the second group of transcripts or translation products is found to be higher in the first sample than in the second sample.

In another embodiment of the invention a method is provided for evaluating carcinogenicity of an agent to human prostate cells. The level of expression of at least one transcript or its translation product from a first or a second group of RNA transcripts is compared. The level of expression in a first sample of human prostate cells contacted with a test agent is compared to level of expression in a second sample of human prostate cells not contacted with the test agent. The first group of RNA transcripts consists of transcripts of genes selected from the group consisting of genes ranked 1-5, 7-8, 10-13, 15-17,19-20,22, 24-25, 27-28, 31, 34-36, 38, 39, 40-43, 45-61, and 62 as shown in Table 4, and the second group of RNA transcript consists of transcripts of genes selected from the group consisting of genes ranked 1, 3, 5-21, and 22 as shown in Table 3. An agent is a potential carcinogen to human prostate cells if it decreases the level of expression of at least one of the genes of the first group, or increases the level of expression of at least one of the genes in the second group.

In another embodiment of the invention a method is provided for slowing progression of prostate cancer in a patient. A polynucleotide is administered to prostate cancer cells of the patient. The polynucleotde comprises a coding sequence of a gene selected from the group consisting of genes ranked 1-5, 7-8,10-13, 15-17, 19-20,22, 24-25, 27-28, 31, 34-36,38,39,40-43,45-61, and 62 as shown in Table 4. The gene is expressed in the prostate cancer cells and slows progression of prostate cancer in the patient.

In another embodiment of the invention a method is provided for slowing progression of prostate cancer in a patient. An antisense construct is administered to prostate cancer cells of a patient. The antisense construct comprises at least 12 nucleotides of a coding sequence of a gene selected from the group consisting of gene numbers 1, 3, 5-21, and 22 as shown in Table 3. The coding sequence is in a 3′ to 5′ orientation with respect to a promoter that controls its expression, and an antisense RNA is expressed in cells of the cancer, slowing progression of prostate cancer in the patient.

In another embodiment of the invention a method is provided for slowing progression of prostate cancer in a patient. In this method an antibody is administered to prostate cancer cells in a patient. The antibody specifically binds to a protein expressed from a gene selected from the group consisting of genes ranked 1, 3, 5-21, and 22 as shown in Table 3. The antibody binds to the protein and slows progression of prostate cancer in the patient.

In another embodiment of the invention a method is provided for screening candidate drugs useful in the treatment of prostate cancer. A prostate cancer cell is contacted with a test substance. Expression of a transcript or translation product of a gene from a first or second group is monitored. The first group consists of genes ranked 1-5, 7-8, 10-13,15-17, 19-20, 22, 24-25, 27-28, 31, 34-36, 38, 39, 40-43, 45-61, and 62 as shown in Table 4 and the second group consists of genes ranked 1, 3, 5-21, and 22 as shown in Table 3. A test substance is identified as a potential drug useful for treating prostate cancer if it increases expression of at least one of the genes in the first group or decreases expression of at least one of the genes in the second group.

In another embodiment of the invention a method is provided for diagnosing prostate cancer in a patient. The level of expression of at least one RNA transcript or its translation product in a test sample of prostate tissue is compared to the level of expression of the at least one RNA transcript or translation product in a control sample of prostate tissue. The test sample of prostate tissue is suspected of being neoplastic and the control sample is nonmalignant human prostate tissue. At least one RNA transcript or its translation product is selected from a first or a second group of RNA transcripts or translation products. The first group of RNA transcripts consists of transcripts of genes selected from the group consisting of genes ranked 1-5, 7-8, 10-13, 15-17,19-20,22, 24-25,27-28, 31, 34-36,38, 39,40-43, 45-61, and 62. The second group of RNA transcripts consists of transcripts of genes selected from the group consisting of genes ranked 1, 3, 5-21, and 22 as shown in Table 3. The test sample is identified as cancerous when expression of at least one of the first group of RNA transcripts or translation products is found to be lower in the test sample than in the control sample, or expression of at least one of the second group of transcripts or translation products is found to be higher in the test sample than in the control sample.

In another embodiment of the invention an array of nucleic acid molecules is provided. The nucleic acid molecules of the array comprise a set of members having distinct sequences, and each member is fixed at a distinct location on the array. At least 10% of the members on the array comprise at least 15 contiguous nucleotides of genes selected from the group consisting of genes ranked 1-5, 7-8, 10-13, 15-17,19-20, 22, 24-25, 27-28, 31, 34-36, 38, 39, 40-43, 45-61, and 62 as shown in Table 4, and genes ranked 1, 3, 5-21, and 22 as shown in Table 3.

In another embodiment of the invention a method is provided for monitoring or predicting the outcome of prostate cancer in a patient. The level of at least one serum marker is measured in a serum sample of a patient with prostate cancer. The serum marker is a protein expressed from a first or second group of genes. The first group of genes is selected from the group consisting of genes ranked 4, 7,18, 22, 26, 30, 38, 41, 53, and 55 as shown in Table 4. The second group of genes consists of PLA2G7/LDL-phospholipase A2 (U24577).

In another embodiment of the invention a method is provided for diagnosing prostate cancer in a patient. The level of at least one serum marker is measured in a serum sample of a patient suspected of having prostate cancer. The serum marker is a protein expressed from a first or second group of genes. The first group of genes is selected from the group consisting of genes ranked 4, 7,18, 22, 26, 30, 38, 41, 53, and 55 as shown in Table 4. The second group of gene consists of PLA2G7/LDL-phospholipase A2 (U24577). The patient can be identified as having prostate cancer when the level of a serum marker expressed from the first group is found to be reduced in the patient relative to an individual with a nonmalignant prostate or when the level of the serum marker of the second group is found to be increased in the patient relative to an individual with a nonmalignant prostate.

The present inventions thus provide reagents and tools for diagnosing, slowing the progression of, and monitoring and predicting the outcome of prostate cancer in a patient. The present inventions also provide methods for evaluating carcinogenicity of an agent to human prostate cells, and for screening for candidate drugs for treating prostate cancer. Nucleic acid arrays are also provided.

### BRIEF DESCRIPTION OF THE DRAWINGS

FIG. 1 illustrates a flow diagram of data reduction process from −6800 genes to 22 up regulated genes and 64 down regulated genes.

FIG. 2 illustrates hierarchical clustering using expression profiles of 5150 transcripts using the method of cosine correlation of similarity coefficient.

Benign prostatic hyperplasia (BPH) and Gleason grade 4/5 cancer (G 4/5) samples are clustered independently. The only two transition zone cancers that had invaded the peripheral zone form an independent sub-cluster (#6 and #2) within the grade 4/5 cancers.

FIG. 3 illustrates the functional categorization of candidate genes. Wherein the following numbered functional categories represented include: 1. Amino acid metabolism; 2. Apoptosis related; 3. Onocogene/suppressor; 4. Carbohydrate metabolism; 5. Cell cycle; 6. Cell proliferation/differentiation/cell communication; 7. DNA binding; 8. Growth factor; 9. Immune related; 10. Ion channels; 11. Kinase/signaling/G protein; 12. Oxidase; 13. Matrix metalloproteinases; 14. Oxidase; 15. Structural protein; 16. Transcription factor; 17. Transferase; 18. Transport; 19. Others; and 20. Unknown function.

### TABLE LEGENDS

Table 1 shows clinical and histologic details of radical prostatectomy specimens and frozen section in nine men with Gleason grade 4/5 cancers. The symbol a denotes cancer located in transition zone; all others were located in peripheral zone. The symbol ^{b} denotes that samples 9 and 14 (see Table 2) are from the same prostate. The symbol ^{c} denotes a large secondary cancer 2.4 cc, 70% grade 4/5. The average age of the nine men with cancer was 58 years.

Table 2 shows clinical and histologic details of radical prostatectomy specimens in eight men with benign prostatic hyperplasia (BPH). In none of these men was there any cancer in the frozen section of the BPH nodules. Sample 9 (see Table 1) is from the same prostate as sample 14. The average age of the eight men with BPH was 62 years.

Table 3 shows 22 up regulated genes (of 5,150) selected by p-value difference of 0.0005 between nine Gleason grade 4/5 and eight BPH prostate tissues. These genes are ordered by their Fold Changes (FC) of two or greater in comparing the transcript expression levels between Gleason grade 4/5 cancer and BPH. An * denotes genes known to be related to cancer.

Table 4 shows 64 down regulated genes. These genes are ordered by their Fold Changes (FC) of two or greater in comparing the transcript expression levels between BPH and Gleason grade 4/5 cancer. An * denotes genes known to be related to cancer.

Table 5 shows the chromosomal location of 18 up regulated and 63 down regulated genes.

### DETAILED DESCRIPTION OF THE INVENTION

A specific differential pattern of gene expression between benign prostate hyperplasia (BPH) and Gleason 4/5 carcinoma has been discovered. The differentially expressed genes may be used to diagnose prostate carcinoma, predict the outcome of prostate carcinoma, and slow the progression of prostate cancer. Prostate carcinoma may be diagnosed, or the outcome of prostate carcinoma may be predicted, by comparing levels of RNA transcripts or translation products, or comparing levels of serum markers between samples. Administering antibodies, antisense, or genes of the invention may slow the progression of prostate cancer. The differentially expressed genes may also be used to evaluate the carcinogenicity of an agent to human prostate cells, to screen for drugs to treat prostate carcinoma, and on nucleic acid arrays.

Many methods of the invention compare the level of expression of RNA transcripts or translation products. Measuring the level of expression of these RNA transcripts or translation products may be performed by any means known in the art. Examples of methods to determine protein levels include immunochemistry such as radioimmunoassay, Western blotting, and immunohistochemistry. RNA levels may be measured using an array of oligonucleotide probes immobilized on a solid support. Northern blotting and in situ hybridization may also be performed to determine levels of RNA transcripts in samples. Comparison can be done by observation, by calculation, by optical detectors, or by computers, or any other means.

The levels of expression of these RNA transcripts or translation products are compared in methods of the invention, for instance, between different samples of prostate tissue. Higher levels of expression are defined as any statistically significant increase in expression of the RNA transcripts or translation products from one prostate sample relative to another prostate sample. The increase in expression may be, for example, 1.5-, 2-, 3-, 4.0-, 5-, or 10-fold higher. Lower levels of expression are defined as any statistically significant decrease in expression of the RNA transcripts or translation products from one prostate sample relative to another prostate sample. The decrease in expression may be, for example, 1.5-, 2-, 3-, 4.0-, 5-, or 10-fold lower.

The outcome of prostate cancer in a patient can be predicted. The level of expression of at least one RNA transcript or its translation product, in a first sample of prostate tissue that is neoplastic is compared to a second sample of human prostate tissue that is nonmalignant. The transcript is a transcript of a gene selected from the first group consisting of genes ranked 1-5, 7-8, 10-13, 15-17,19-20, 22, 24-25, 27-28, 31, 34-36, 38, 39, 40-43, 45-61, and 62 as shown in Table 4 or the transcript is a transcript of a gene selected from a second group consisting of genes ranked 1, 3, 5-21, and 22 as shown in Table 3. The patient is identified as having a poor outcome when expression is found to be lower in the first sample than in the second sample for the genes of the first group, or when expression is found to be higher in the first sample than in the second sample for the genes of the second group. Neoplastic prostate tissue exhibits abnormal histology that is consistent with cancerous cell growth at any stage of disease. The neoplastic tissue may be characterized as any of Gleason grades 1, 2, 3, 4, or 5. Neoplastic cells of Gleason grade 4/5 are particularly useful. Nonmalignant prostate tissue is free of any pathologically detectable cancer. The nonmalignant prostate tissue may be free of any prostate disease or abnormal growth. The nonmalignant tissue may also be benign prostate hyperplasia (BPH) tissue.

A poor outcome is the result of progression of the neoplastic tissue from one Gleason grade to a higher Gleason grade. A poor outcome is associated with Gleason 4/5 prostate cancer. Even no change in marker pattern from a prior measurement may be characterized as a poor outcome.

Transcripts or translation products may be compared of at least 2, 5, 10, 20, 30, or 49 of the genes in the first group. Transcripts or translation products may be compared of at least 2, 5, 10, or 20 of the genes in the second group. Members of one or both groups can be compared. The information supplied by the two groups of genes may provide increased confidence in the findings. For example, transcripts or translation products of at least 2, 5, 10, or 20 transcripts in each of the first and second groups may be compared. Transcripts or translation products may also be compared of at least 30 transcripts or translation products in the first group and 20 transcripts or translation products in the second group, or of at least 40 transcripts or translation products in the first group and 20 transcripts or translation products in the second group, or of at least 49 transcripts or translation products in the first group and 20 transcripts or translation products in the second group.

Carcinogenicity of an agent to human prostate cells can be evaluated using the genes involved in prostate cancer. Level of expression of at least one transcript or its translation product from a first or a second group of RNA transcripts is compared. A first sample of human prostate cells is contacted with a test agent and a second sample of human prostate cells is not contacted with the test agent. The levels of expression of at least 1, 2, 5, 10, 20, 50, 60, or 69 of the RNA transcripts or translation products may be compared. The first group of RNA transcripts consists of transcripts of genes selected from the group consisting of genes ranked 1-5, 7-8, 10-13, 15-17, 19-20, 22, 24-25, 27-28, 31, 34-36, 38, 39, 40-43, 45-61, and 62 as shown in Table 4, and the second group of RNA transcript consists of transcripts of genes selected from the group consisting of genes ranked 1, 3, 5-21, and 22 as shown in Table 3. An agent is identified as a potential carcinogen to human prostate cells if it decreases the level of expression of at least one of the genes of the first group, or increases the level of expression of at least one of the genes in the second group.

Test agents may include any compound either associated or not previously associated with carcinogenesis of any cell type. Nonlimiting examples of test agents include chemical compounds that mutagenize DNA, or environmental factors such as ultraviolet light. Test agents also include pesticides, ionizing radiation, cigarette smoke, and other agents known in the art. Test agents may also be proteins normally found in the human body that cause abnormal changes in prostate cells or environmental factors known to induce tumors in other human tissues but that have not yet been associated with prostate cancer.

Any level of changed expression that may be induced in prostate cells identifies carcinogenicity. Desirably the change in expression is statistically significant and includes a change of at least 50%, 200%, 300%, 400%, or 500%.

Nonmalignant human prostate cells may be isolated from any human prostate free of malignant disease. The human prostate cells may also be human prostate cells that have been maintained in culture, such as transformed cell lines, that are nonmalignant. Nonmalignant includes both disease free and benign prostate hyperplasia.

In order to slow progression of prostate cancer in a patient one can administer to the patient a polynucleotide comprising a coding sequence of a gene selected from the group consisting of genes ranked 1-5, 7-8, 10-13, 15-17, 19-20, 22, 24-25, 27-28, 31, 34-36, 38, 39, 40-43, 45-61, and 62 as shown in Table 4. Administration of the gene slows progression of prostate cancer in the patient.

An antisense construct can be administered to prostate cells of a patient. The antisense construct comprises at least 12 nucleotides of a coding sequence of a gene selected from the group consisting of genes ranked 1, 3, 5-21, and 22 as shown in Table 3. The coding sequence is in a 3′ to 5′ orientation with respect to a promoter which controls its expression, whereby an antisense RNA is expressed in cells of the cancer and progression of prostate cancer in the patient is slowed. Alternatively, antisense oligonucleotides that bind to mRNA can be directly administered without a vector.

An antibody that specifically binds to a protein expressed from a gene selected from the group consisting of genes ranked 1, 3, 5-21, and 22 as shown in Table 3 can be administered to a patient. The antibody binds to the protein and progression of prostate cancer is slowed in the patient.

Slowing progression of prostate cancer in a patient includes reduction of the rate of growth of prostate tumors at the prostate of the patient. Slowing progression of prostate cancer in a patient also includes a reduction in the rate of spread of the prostate tumor from the prostate to other sites in a patient. Furthermore, slowing progression of prostate cancer includes a reduction in the size of the prostate tumor, or the prevention of the spread of the prostate cancer in the patient. Any amount or type of reduced progression of the prostate cancer is desirable.

A polynucleotide includes all or a portion of the coding sequence of any of the genes identified. The gene segment may be linear, cloned into a plasmid, cloned into a human artificial chromosome, or cloned into another vector. Vectors also include viruses that are used for gene delivery. Viruses include herpes simplex virus, adenovirus, adeno-associated virus, or a retrovirus. The adenoviral vector may be helper virus dependent. The naked DNA may also be injected, or may be associated with lipid preparations, such as liposomes.

Any nucleic acid that binds to the identified genes or the RNA transcripts of the identified genes and prevents expression of their products can be used as a therapeutic antisense reagent. The antisense may be an oligonucleotide or ribozyme, or any other such polynucleotide known in the art. The antisense RNA will bind anywhere along the identified genes or RNA transcripts, including within the coding region or regulatory region of the gene sequence. The antisense also does not have to be perfectly complementary to the sequence of the identified genes or transcripts. It may also be of any effective length. The antisense polynucleotide may be at least 12, 15, 18, 21, 24, 27, 28, 29, or 30 bases in length. The antisense may or may not be driven by a promoter.

A promoter is a sequence that drives expression of RNA. Any of the suitable promoters known in the art may be used. The promoter may be a strong promoter derived from a virus, such as the mouse mammary tumor virus promoter, or Rous sarcoma virus promoter. The promoter may also be constitutive promoter that is active in all tissues, or may be a tissue specific promoter. Preferably, a tissue-specific promoter is a promoter specific to the prostate. Several non-limiting examples of such promoters are the prostate specific antigen (PSA) promoter, the probasin (PB) promoter, and the prostate specific membrane antigen promoter.

Any modifications, such as the introduction of phosphorothioate bonds in the polynucleotides, may be made to increase the half-life of antisense polynucleotides in the patient. Other non-phosphodiester internucleotide linkages that may be introduced into the polynucleotides include phosphorodithioate, alkylphosphonate, alkylphosphonothioate, alkylphosphonate, phosphoramidate, phosphate ester, carbamate, acetamidate, carboxymethyl esters, carbonates, and phosphate triester. The bases or sugars of the nucleotides may be modified as well. For instance, arabinose may be substituted for ribose in the antisense oligonucleotide.

Administration of the gene or antisense construct can be by any acceptable means in the art. These include injection of the nucleic acids systemically into the bloodstream of the patient or into the prostate tumor directly. The nucleotides may also be administered topically or orally. The gene or antisense construct may be formulated with an excipient such as a carbohydrate or protein filler, starch, cellulose, gums, or proteins such as gelatin and collagen. The gene or antisense construct may be formulated in an aqueous solution. Preferably the solution is in a physiologically compatible buffer. Acceptable buffers include Hanks' solution, Ringer's solution, or physiologically buffered saline.

Antibodies that specifically bind to any epitope of the indicated proteins will slow the progression of the prostate cancer. The antibodies may be of any isotype, for example, IgM, IgD, IgG, IgE, or IgA. The antibodies may be full-length or may be a fragment or derivative thereof. For instance, the antibodies may be only the single chain variable domain, or fragments of the single chain variable domain. The antibodies may be in a monoclonal or a polyclonal preparation. The antibodies may also be produced from any source and may be conjugated to toxins or other foreign moieties. The antibodies may be produced using the hybridoma technique or the human B-cell hybridoma technique. They may also be produced by injection of peptide into animals such as guinea pigs, rabbits, or mice. Antibodies preferably bind to serum markers or cell surface proteins. The antibodies can be humanized or chimeric.

Candidate drugs can be screened for those useful in the treatment of prostate cancer. Prostate cancer cells can be contacted with a test substance. Expression of a transcript or its translation product from a first or second group is monitored. The transcript is of a gene selected from a first group consisting of genes ranked 1-5, 7-8, 10-13, 15-17, 19-20, 22, 24-25, 27-28, 31, 34-36, 38, 39, 40-43, 45-61, and 62 as shown in Table 4 and genes from a second group consisting of genes ranked 1, 3, 5-21, and 22 as shown in Table 3. A test substance is identified as a candidate drug useful for treating prostate cancer if it increases expression of at least one of the genes in the first group or decreases expression of at least one of the genes in the second group.

A test substance can be a pharmacologic agent already known in the art for another purpose, or an agent that has not yet been identified for any pharmacologic purpose. It may be a naturally occurring molecule or a molecule developed through combinatorial chemistry or using rational drug design. A test substance also may be nucleic acid molecules or proteins.

These may or may not be found in nature. Test substances are identified as candidate drugs if they increase expression of at least one of the genes in the group consisting of genes ranked 1-5, 7-8, 10-13, 15-17, 19-20, 22, 24-25, 27-28, 31, 34-36, 38, 39, 40-43, 45-61, and 62 as shown in Table 4 or decrease expression of at least one of the genes in the group consisting of genes ranked 1, 3, 5-21, and 22 as shown in Table 3. Candidate drugs, as used herein, are drugs that are potentially useful for treating cancer. It is contemplated that further tests may be needed to evaluate their clinical potential after identification in the method. Such tests include animal models and toxicity testing, inter alia.

Prostate cancer can be diagnosed by comparing the level of expression of at least one RNA transcript or its translation product from a first or a second group of RNA transcripts. The first group of RNA transcripts consists of transcripts of genes selected from the group consisting of genes ranked 1-5, 7-8, 10-13, 15-17, 19-20, 22, 24-25, 27-28, 31, 34-36, 38, 39, 40-43, 45-61, and 62. The second group of RNA transcripts consists of transcripts of genes selected from the group consisting of genes ranked 1, 3, 5-21, and 22 as shown in Table 3. The test sample is identified as cancerous when expression of at least one of the first group of RNA transcript or translation products is found to be lower in the test sample than in the control sample, or expression of at least one of the second group of transcripts or translation products is found to be higher in the test sample than in the control sample. Any number of transcripts can be compared.

For example, the level of expression of at least 1, 2, 5, 10, 20, 30, or 49 transcripts of the first group may be compared. Alternatively, the level of expression of at least 1, 2, 5, 10, or 20 transcripts of the second group may be compared. Alternatively, at least 2, 5, 10, or 20 transcripts of each of the first and second groups are compared. Alternatively, at least 30 transcripts or translation products in the first group and 20 transcripts or translation products in the second group, or 40 transcripts or translation products in the first group and 20 transcripts or translation products in the second group, or 49 transcripts or translation products in the first group and 20 transcripts or translation products in the second group are compared. The at least one transcript or translation product of the first group preferably comprises the transcript of the gene maspin. The at least one RNA transcript or its translation product of the second group of RNA transcripts preferably includes hepsin.

Arrays of nucleic acids comprise nucleic acid molecules that have distinct sequences that are fixed at distinct locations on the array. At least 10% of the molecules on the array also comprise at least 15 contiguous nucleotides of gene selected from the group consisting of genes ranked 1-5, 7-8, 10-13, 15-17, 19-20, 22, 24-25, 27-28, 31, 34-36, 38, 39, 40-43, 45-61, and 62 as shown in Table 4, and genes ranked 1, 3, 5-21, and 22 as shown in Table 3. At least 10%, 20%, 30%, 40%, 50%, 60%, 75%, 80%, or 90% of the molecules on the array may also comprise at least 15 contiguous nucleotides of any of the indicated genes of Tables 3 and 4. The GeneChip® system (Affymetrix, Santa Clara, Calif.) is a particularly suitable array, however, it will be apparent to those of skill in the art that any similar systems or other effectively equivalent detection methods can also be used. Nucleotide arrays are disclosed in U.S. Pat. Nos. 5,510,270, 5,744,305, 5,837,832, and 6,197,506, each of which is incorporated by reference. The nucleotide array is typically made up of a support on which probes are arranged. The support may be a chip, slide, beads, glass, or any other substrate known in the art. Oligonucleotide probes are immobilized on the solid support for analysis of the target sequence or sequences. For methods of attaching a molecule with a reactive site to a support see U.S. Pat. No. 6,022,963, which is herein incorporated by reference in its entirety. For probes that may be used with arrays see U.S. Pat. No. 6,156,501, which is herein incorporated by reference in its entirety. For methods of monitoring expression with arrays see U.S. Pat. Nos. 5,925,525 and 6,040,138, each of which are incorporated herein by reference.

The outcome of prostate cancer can be predicted in a patient. The level of at least one serum marker in a patient with prostate cancer can be measured. The serum marker is a protein expressed from a gene of a first or second group. The genes of the first group are selected from the group consisting of genes ranked 4, 7,18, 22, 26, 30, 38, 41, 53, and 55 as shown in Table 4. The gene of the second group consists of PLA2G7/LDL-phospholipase A2 (U24577). A serum marker is a protein that is secreted from cells and that is detected in the serum of the patient. The serum marker may be detected by any means known in the art, including measurement with an antibody. Techniques that may be used to detect the serum marker include enzyme-linked immunosorbent assay, sandwich immunoassay, Western blot analysis or other immunoassays. The protein may also be immunoprecipitated and run through a polyacrylamide gel. An individual with a nonmalignant prostate is an individual free of any malignant prostate disease. It is contemplated that the individual may have benign prostate hyperplasia.

Prostate cancer in a patient can be diagnosed using the disclosed markers. The level of at least one serum marker in a patient can be measured. The serum marker is a protein expressed from a gene of a first or second group. The genes of the first group are selected from the group consisting of genes ranked 4, 7, 18, 22, 26, 30, 38, 41, 53, and 55 as shown in Table 4. The gene of the second group consists of PLA2G7/LDL-phospholipase A2 (U24577). The patient is identified as having prostate cancer when the level of a serum marker from the first group is found to be reduced in the patient relative to an individual with a nonmalignant prostate or the level of the serum marker from the second group is found to be increased in the patient relative to an individual with a nonmalignant prostate. The level of serum marker can be determined with an antibody. As indicated previously, techniques that may be used to detect the serum marker include enzyme-linked immunosorbent assay, sandwich immunoassay, Western blot analysis or other immunoassays. The protein may also be immunoprecipitated and run through a polyacrylamide gel.

The above disclosure generally describes the present invention. A more complete understanding can be obtained by reference to the following specific examples that are provided herein for purposes of illustration only, and are not intended to limit the scope of the invention. All references cited in this application are expressly incorporated for all purposes.

### EXAMPLES

Example 1

### Limitations Imposed by Heterogeneous Zones in the Prostate

Because Gleason grade 4/5 cancer is the primary cause of failure to cure prostate cancer, the molecular profiles of this high-grade cancer were examined in search of potentially new therapeutic interventions as well as better serum markers than prostate-specific antigen.

It is thought that this is the first effort to characterize the up (Table 3) and down (Table 4) regulated genes specifically in Gleason grade 4/5 cancer. Gene expression levels in BPH were used as a control for increased and decreased expression. Eighty-six genes with p-value differences of at least <0.0005 in expression between grade 4/5 cancer and BPH have been found. These genes could play a substantial role in finding tumor markers for grade 4/5 cancer, as well as potentially elucidating some new therapeutic approaches. Most of these genes have not been previously reported as having a relationship to prostate cancer, and are all the more remarkable when one considers the tissue heterogeneity of the three histologically different prostate zones confined within a single capsule enclosing prostates weighing only 35-65 grams with a mean of 46 grams (Tables 1 and 2).

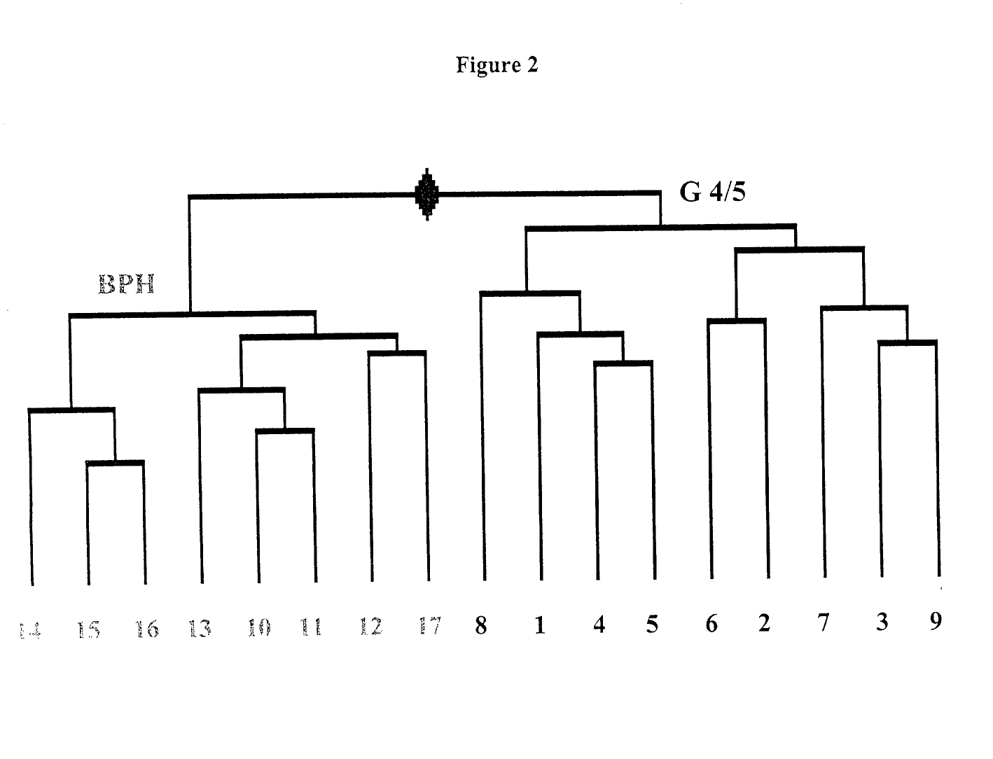

The insights gained into Gleason grade 4/5 cancer in comparison to BPH in this research identified several new markers as well as new therapeutic modalities. The central zone of the prostate is highly resistant to developing prostate cancer. McNeal, J. E., Regional Morphology and Pathology of the Prostate. Am J Clin Path, 49:347-57, 1968, herein incorporated by reference in its entirety. Comparing grade 4/5 cancer to normal central zone may lead to even more interesting genes. Since transition zone cancers have a far better prognosis than the much more common peripheral zone cancers, even when matched by similar cancer volumes and grade, the distinction between these cancers at the molecular level might lead to new insights into these two highly different but common prostate cancers. Chang, S. S., O'Keefe, D. S., Bacich, D. J., Reuter, V. E., Heston, W. D. W., Gaudm, P. B., Prostate-specific membrane antigen is produced in tumor-associated neovasculature, Clin Cancer Res, 5: 2674-2681,1999, herein incorporated by reference in its entirety. It is of some interest in FIG. 2 that the only two grade 4/5 cancers in the peripheral zone that originated in the transition zone (Table 1) form a sub-cluster in FIG. 2 (No. 6 and No. 2).

The peripheral zone dysplasia directly gives rise to Gleason grade 3 cancers. McNeal, J. E., Villers, A., Redwine, E. A., Freiha, F. S., Stamey, T. A., Microcarcinoma in the prostate: Its association with duct-acinar dysplasia. Human Pathology, 22:644-652,1991, herein incorporated by reference in its entirety. Thus, the final molecular understanding of prostate cancer must include the evolutionary genetic events from normal PZ tissue dysplasia grade 3 cancer grade 4 cancer. This work characterizes the latter event upon which cure by radical prostatectomy appears to depend. Stamey, T. A., McNeal, J. E., Yemoto, C. M., Sigal, B. M., Johnstone, I. M., Biological determinants of cancer progression in men with prostate cancer, JAMA, 281: 1395-400,1999, herein incorporated by reference in its entirety.

Probe arrays were used to measure gene expression levels in about 6,800 human genes in Gleason grade 4/5 cancer from radical prostatectomy specimens. Nodules of BPH were used as controls for several reasons, the most important of which is the histologic heterogeneous nature of the prostate. The prostate is composed of three distinct zones: the peripheral zone, from which 80% of all prostate cancers arise; the central zone, which appears resistant to cancer origin but contains almost half of all the prosaic epithelial cells in an average adult male under 40 years old; and the transition zone (TZ) in which BPH arises, sometimes accompanied by the remaining 20% of prostate cancers. McNeal, J. E., Regional Morphology and Pathology of the Prostate, Am J Clin Path, 49:347-57,1968 and McNeal, J. E., Prostate, In Histology for Pathologists, 2nd edition, Edited by Stephen S. Sternberg, Philadelphia: Lippincoft-Raven Publishers, chapter 42, 997-1017, 1997, herein incorporated by reference in their entirety. Other reasons for using nodules of BPH as control cells for gene expression analysis include the histologic identity of PZ epithelial cells and TZ epithelial cells when viewed with the high power of the microscope although they are readily distinguishable with the low-power field by the incorporation of TZ cells into a pattern of nodular architecture. More importantly, it is observed that almost all available antibodies for studying prostate epithelium appear to stain both PZ and TZ epithelial types equivalently. Finally, a complete transverse section across the mid-gland of any prostate >50 grams in size is almost certain to reveal some nodules of BPH. While “normal” PZ cells would be ideal as control epithelium for PZ grade 4/5 cancer, unfortunately epithelial atrophy and dysplasia, the latter of which gives rise to Gleason grade 3 cancer in the PZ, are very common in prostates from men >50 years old. McNeal, J. E., Villers, A., Redwine, E. A., Freiha, F. S., Stamey, T. A., Microcarcinoma in the prostate: Its association with duct-acinar dysplasia. Human Pathology, 22:644-652,1991, herein incorporated by reference in its entirety. For these reasons, the gene transcripts in nine men with Gleason grade 4/5 cancer were compared to eight men with nodules of BPH. In only one instance was a Gleason grade 4/5 cancer and BPH obtained from the same individual; these two samples were histologically similar to the other grade 4/5 and BPH samples.

Tissue Processing

Samples of prostatic tissue were obtained within 15 minutes of intraoperative interruption of the blood supply to the prostate, covered with O.C.T. Compound 4583 (Tissue-Tek®, Sakura Finetek, Torrance, Calif., USA) in frozen section molds, placed in liquid nitrogen for 15 minutes, and transferred to a storage freezer at −70° C. After transfer to a Leica CM1850 cryostat, 5 μm sections were cut for hemotoxylin and eosin cover-glass examination, followed by ten 60 μm sections for trizol (TRIZOL® Reagent, Molecular Research Center, Cincinnati, Ohio, USA) extraction of RNA. If the shape of the tissue section changed on the cryostat during removal of the ten 60 μm sections, further 5 μm sections were examined to compare with the first 5 μm sections. If the tissue of interest in the 5 μm sections was reasonably uniform between the first and last 5 μm sections, then procession to RNA extraction of the 60 μm sections was done. If histologic areas of tissue were foreign to our point of interest, the contaminating area was removed with a cold knife in the cryostat, trimmed the excess OCT, and proceeded with trizol extraction of the whole tissue RNA, discarding the ten 60-micron sections. Patient age, preoperative serum PSA levels, and histologic details of the 17 prostates are provided in Tables 1 and 2 for the radical prostatectomy specimens and for the frozen tissue samples submitted for RNA extraction.

Isolation of Total RNA

Trimmed prostate tissue blocks or the ten 60-micron sections were homogenized with trizol reagent using a power homogenizer (Polytron) for 10 minutes and incubated at room temperature for five minutes to allow complete dissociation of nucleobinding proteins. To the homogenized samples, 0.2 ml was added, of chloroform per 1.0 ml of trizol reagent, which was vigorously shaken by hand for 15 seconds and incubated at room temperature for three minutes. The samples were then centrifuged at 12,000×g for 15 minutes in a cold room; 0.6 ml of the colorless upper aqueous phase (that contained tissue total RNA) was transferred to a fresh tube. Isopropyl alcohol (0.5 ml) and 1 μl of glycogen were used to precipitate RNA at room temperature. After 15 minutes, the RNA pellets were obtained by centrifugation at 12K×g for 10 minutes in a cold room, washed twice with 75% ethanol by vortexing, followed by centrifugation. Total RNA was further purified using the RNeasy® Mini Kit (Qiagen, Inc., Valencia, Calif., USA) according to the manufacturer's instructions.

cDNA Synthesis and Labeling

Double-strand cDNA was synthesized from total RNA; labeled cRNA was prepared from cDNA, as described by Mahadevappa and Warrmgton and applied to HuGeneFI® probe arrays representing 6,800 genes. Mahadevappa, M., Warrington, J. A., A high density probe array sample preparation method using 10-100 fold fewer cells. Nature Biotech, 17:1134-1136,1999, herein incorporated by reference in its entirety. The arrays were synthesized using light-directed combinatorial chemistry, as described by Fodor et al. Fodor, S. P. A., Read, J. L., Pirrung, M. C., Stryer, L., Lu, A. T., Solas, D., Light-directed spatially addressable parallel chemical synthesis, Science, 251: 713-844,1991 and Fodor, S. P. A., Rava, R. P., Huang, X. C., Pease, A. C., Holmes, C. P., and Adams, C. L., Multiplexed biochemical assays with biological chips, Science, 364: 555-556, 1993, which are herein incorporated by reference in their entirety.

Fragmentation, Array Hybridization, and Scanning

All procedures were carried out as described by Warrington et al. Warrington, J. A., Nair, A., Mahadevappa, M., Tsyganskaya, M., Comparison of human adult and fetal expression and identification of 535 housekeeping/maintenance genes, Phys Genomics, 2: 143-147, 2000, herein incorporated by reference in its entirety.

Sample Quality

Sample quality was assessed by agarose gel electrophoresis and spectrophotometry (A₂₆₀/A₂₈₀ ratio) using aliquots of total RNA to evaluate whether or not the RNA was of sufficient quality to continue. If the total RNA appeared intact, the samples were prepared and hybridized to the GeneChip® Test3 Array (Affymetrix, Inc., Santa Clara, Calif.) to determine the ratio of 3′ and 5′ GAPDH (glyceraldehydes 3-phosphate dehydrogenase) transcript levels and finally to the HuGeneFI arrays. Of the 22 samples collected, 17 met the sample quality criteria of a GAPDH ratio less than 3 and more than 40% of the transcripts represented on the array.

The gene expressions in fresh frozen tissues from nine men with Gleason grade 4/5 cancer was compared to eight men with benign prostatic hyperplasia (BPH), all undergoing radical retropubic prostatectomy. Labeled cRNA from each of the 17 tissues was applied to HuGeneFL® probe arrays representing 6,800 genes (Affymetrix, Inc., Santa Clara, Calif.). The histologic characteristics of the nine prostates from which the grade 4/5 cancers were selected are shown in Table 1 and the eight prostates from which the BPH tissue samples were obtained are shown in Table 2. Also shown in Table 1 is the histologic information from the frozen section cover glass preparations of the specific prostatic tissue from which RNA was extracted from the nine grade 4/5 cancers. None of the BPH samples contained any contaminating cancer on frozen section examination. Cluster analysis clearly segregates the BPH from the grade 4/5 cancers (FIG. 2).

### Example 2

Data Analysis and Data Reduction

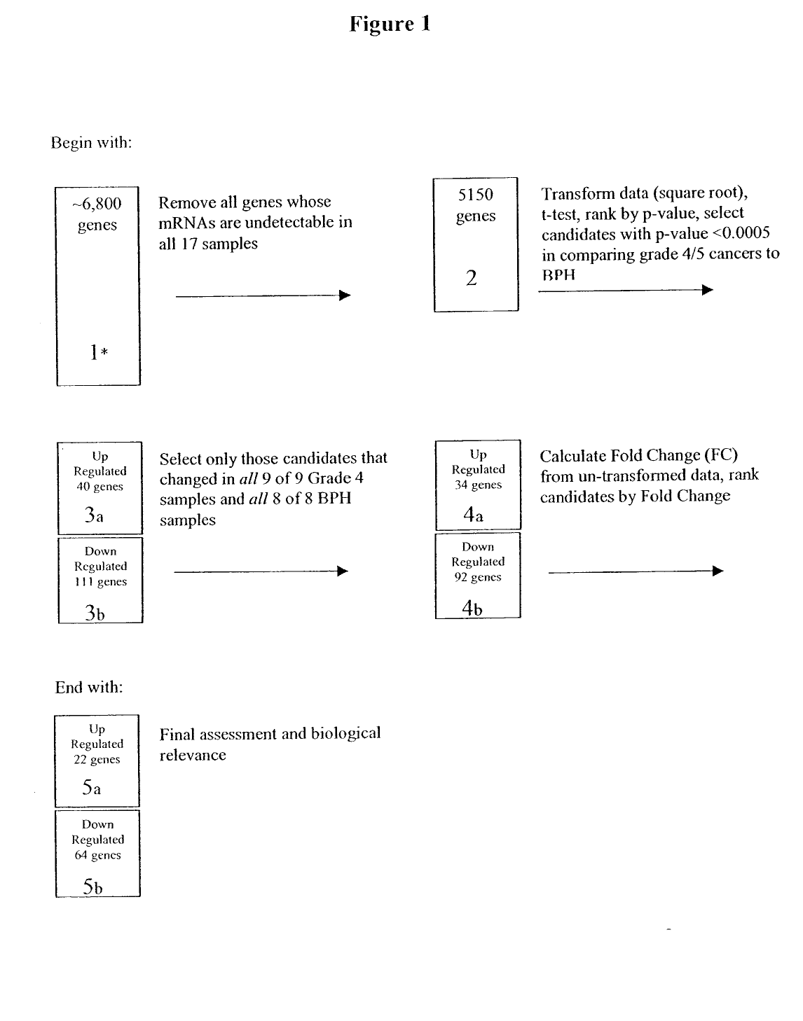

The primary purpose of data analysis in gene array experiments is data reduction, that is, to move from a large number of data points of ˜115,000 (˜6,800 genes×17 tissues) to a smaller group of more significant data points (in this case, <100). FIG. 1 delineates the data reduction steps. To accomplish this, several software tools were used for data analysis, including Microsoft Access and Microsoft Excel (Redmond, Wash. 98052-6399) and Affymetrix Microarray Suite (Santa Clara, Calif. 95051). The ˜6,800 human genes represented on the HuGeneFL® probe array are comprised of probes of single-stranded DNA oligonucleotides 25 bases long, designed to be complementary to a specific sequence of genetic information. Hundreds of thousands to millions of copies of each probe inhabit a probe cell and each cell is a member of a probe pair. Half of that probe pair is comprised of cells that contain exact copies of the DNA sequence, a “Perfect Match”; the companion cell in the probe pair contains copies of the sequence that are altered only at the 13^{th} base, a “Mismatch,” which serves as a control for the Perfect Match sequences. There are 16-20 probe pairs per probe set and each probe set represents one gene. The probe sets are measured for fluorescence, which is proportional to the degree of hybridization between the labeled cRNA from our tissue sample and the DNA on the chip. An average of the differences in fluorescence between the Perfect Match and Mismatch pairs is calculated; this “Average Difference” value is critical and is used in all subsequent calculations for up and down regulation of each gene. Several other values are calculated, one of which, an assessment of whether mRNAs are present, absent, or marginal (“Absolute Call”, is used in other calculations). Warrington, J., Dee, S., Trulson, M., Large-scale genomic analysis using Affymetrix GeneChip® probe arrays. In: Microarray Biochip Technology. Edited by M. Schena, Naick, Mass.: Easton Publishing; chapter 6, 119-148, 2000, herein incorporated by reference in its entirety. All probe sets that were undetectable in all nine cancers and eight BPH samples were removed and the data set with descriptive statistics was examined.

As expected, our gene expression values were highly skewed with large positive and negative tails, such that the mean exceeded the median by 3.4 times, resulting in a nonparametric distribution. By taking the square root of all values, the data set was transformed into a parametric distribution in which the mean was only 1.06 times the median.

Statistical analysis and subsequent ranking were carried out using Student t-test (unpaired, two-tailed, equal variance). Only up and down regulated genes with a p-value difference in fluorescence between grade 4/5 cancer and BPH of <0.0005 were selected, which reduced the data set to 40 up regulated genes and 111 down regulated genes (FIG. 1). To evaluate the expected number of chance candidates (i.e., false positives) the number of transcripts with a p-value equal to one were counted. Those transcripts with p-values between 0.99 and 1 should represent only chance candidates. There were 19 chance candidates per 0.01 interval of p-values, or 0 chance candidates between 0 and 0.0005. Additionally, two-dimensional and multidimensional clustering patterns were carried out using GenExplore (Applied Maths, Kortrijk, Belgium) and MATLAB (MathWorks, Natick, Mass.). A threshold was applied that eliminated all genes that were not increased or decreased by at least 2 times (2 fold change) in a comparison of every one of the BPH and grade 4/5 tissues and ranked them in terms of up and down regulation. Twenty-two up regulated and 64 down regulated genes met this criterion (FIG. 1). This selection was confirmed by using the recently published technique of Tusher, Tibshirani and Chu. Tusher, V. G., Tibshirani, R., and Chu, G., Significance analysis of microarrays applied to the ionizing radiation response, Proc Natl Acad Sci USA, 98: 51165121,2001, herein incorporated by reference in its entirety. All of our 22 up regulated genes appeared in the first 10% of their “positive significant genes” list and all 64 down regulated genes appeared in the first 13% of their “negative significant genes” list.

After removing all genes undetectable in both BPH and grade 4/5 cancers and transforming the data into a parametric distribution, only those up and down regulated genes with a p-value difference in fluorescence between grade 4/5 cancer and BPH of <0.0005 were chosen; this reduced the data set to 40 up regulated and 111 down regulated genes. All genes that were not expressed in every one of the eight BPH and nine grade 4/5 tissues were eliminated, which produced a final set of 86 genes, 22 up regulated and 64 down regulated.

Example 3

### 86 Genes are Differentially Expressed in BPH Prostate and Gleason Grade 4/5 Cancer Samples

Of the 86 genes identified in all eight BPH and all nine cancers studied, 22 were up regulated (Table 3) and 64 were down regulated (Table 4); 40 of these changed by greater than fourfold (9 increasing and 31 decreasing), and 46 changed by at least 2-fold (13 increasing and 33 decreasing).

Cluster analysis cleanly separated men with grade 4/5 cancers from men with BPH. Only 17 of the 86 candidate genes (20%) are known to be prostate cancer-related; 42 (49%) are related to other cancers. The most up regulated gene is Hepsin, a trypsin-like serine protease with its enzyme's catalytic domain oriented extracellularly. Prostate-specific membrane antigen (PSMA) is the second most up regulated gene (all other reports on PSMA have been at the protein level). The genes for both PSA (hK₃) and human glandular kallikrein 2 (hK₂) showed equivalent expression levels 10 times the average of other genes. Complete lists of all the 22 up regulated genes and 64 down regulated genes, together with their locus on the chromosome, are presented in rank order.

Of the 22 most up regulated genes, hepsin is obviously (Table 3) of key interest. It has been most intensely investigated in the cardiovascular field. Wit, Q., Yu, D., Post, J., Halks-Miller, M., Sadler, J. E., Morser, J., Generation and characterization of mice deficient in hepsin, a hepatic transmembrane serine protease, J Clin Invest, 101: 321-326,1998, herein incorporated by reference in its entirety. It is known to be overexpressed in ovarian cancer. Tanimoto, H., Yan, Y., Clarke, J., Hepsin, a cell surface serine protease identified in hepatoma cells, is overexpressed in ovarian cancer. Cancer Res, 57:2884,1997, herein incorporated by reference in its entirety. Hepsin is a type II cell surface trypsin-like serine protease with its enzyme's catalytic domain oriented extracellularly. It is interesting that maspin, a serine protease inhibitor (Table 4) is the fourth most down regulated gene (10-fold change); i.e., maspin is 10 times more expressed in BPH than in grade 4/5 cancer, potentially supporting, rather than inhibiting, the protease activity of hepsin in Gleason grade 4/5 cancer.

Prostate-specific membrane antigen (PSMA), the second most over-expressed gene, is present in prostate tissue and, importantly, in nonprostatic tumor neovasculature. Chang, S. S., O'Keefe, D. S., Bacich, D. J., Reuter, V. E., Heston, W. D. W., Gaudm, P. B., Prostate-specific membrane antigen is produced in tumor-associated neovasculature, Clin Cancer Res, 5: 2674-2681,1999, herein incorporated by reference in its entirety. All earlier reports have been at the protein level. Our paper is the first report that the PSMA gene is highly over-expressed in the prostate and specifically in Gleason grade 4/5 cancer, which may broaden its potential therapeutic applications in the treatment of prostate cancer. In one immunohistochemical study, antibodies to PSMA stained Gleason grade 4/5 cells more intensely than grades 3, 2, and 1. Darson, M. F., Pacelli, A., Roche, P., et al, Human glandular kallikrem 2 (hK₂) expression in prostatic intraepithelial neoplasia and adenocarcinoma: a novel prostate cancer marker. Urol, 49:857, 1997, herein incorporated by reference in its entirety.

Example 4

### PSA and hK₂ are not Differentially Expressed in BPH and Grade 4/5 Cancer

Prostate-specific antigen (PSA) has played a major role in diseases of the prostate. Its biological function as a serine protease is to lyse the gel-like form of the ejaculate in mammals, presumably to free the sperms for fertilization. Lilja, H., A kallikrein-like serine protease in prostatic fluid cleaves the predominant seminal vesicle protein. J Clin Invest, 76:1899, 1985, herein incorporated by reference in its entirety. Total PSA (hK₃) has an 80% homology with human glandular kallikrein 2 (hK₂), which is also expressed in prostate epithelium, and has been reported to be expressed in cancer tissue (at the protein level) at higher levels than t-PSA. Darson, M. F., Pacelli, A., Roche, P., et al, Human glandular kallikrem 2 (hK₂) expression in prostatic intraepithelial neoplasia and adenocarcinoma: a novel prostate cancer marker. Urol, 49:857,1997, herein incorporated by reference in its entirety. Because the probe sets for t-PSA (X07730) and hK₂ (S39329) are among the 6,800 genes on the HuGeneFL®) probe arrays, it is interesting to compare their expressions in our nine grade 4/5 cancers and our eight BPH tissues. Both genes were very highly expressed in all nine cancers and all eight BPH samples with expression levels at least 10 times the average level of other genes, but despite these high levels, there was no difference in gene expression between grade 4/5 cancer and BPH samples. While this does not completely exclude a difference at the protein level, the overall clinical use of hK₂ as a replacement for t-PSA based on relative gene expression levels does not appear promising.

Example 5

### Functional Categories of Genes Over Expressed and Under Expressed in Gleason 4/5 Cancer and BPH

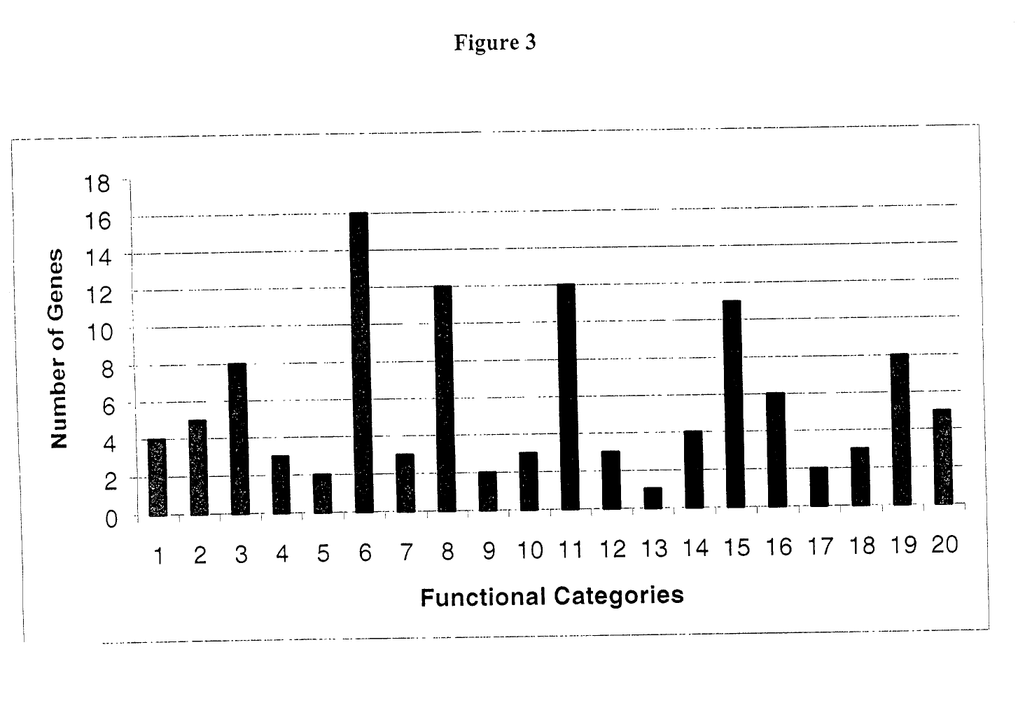

By the literature survey, the set of 86 genes can be divided into 19 functional categories (FIG. 3). The largest category is comprised of 16 genes involved in cell proliferation, communication, and differentiation and includes PLAB (a prostate differentiation factor) and ALCAM (an activated leukocyte cell adhesion molecule). Twelve signal transduction pathway genes were identified, including MacMARCKS, a macrophage cell surface protein which serves as a major substrate for protein kinase C. Eight oncogene/suppressor genes were identified, including PSMA (prostate specific membrane antigen). There are six transcription factor genes, 11 genes classified as having structural functions, and five genes associated with apoptosis, including TRPM-2 (testosteronerepressed prostate message 2). Apoptosis is a common histologic observation in prostate cancer. The 22 most up regulated genes are presented in Table 3 and the 64 most down regulated genes are shown in Table 4. Seventeen of the 86 candidate genes (20%) are known to be prostate cancer-related and are indicated by an asterisk in Tables 3 and 4. Forty-two of 86 genes (49%) are known to be related to other cancers.

Example 6

### Chromosomal Localization of Genes Over Expressed and Under Expressed in Gleason 4/5 Cancer Relative to BPH

It is also interesting and potentially useful to relate the up and down regulated genes to their specific chromosomes (Table 5). Chromosomes 2, 6, 8, 9, 10, 16, 18, and 20 contain only down regulated genes. In fact, half of the down regulated genes have no up regulated chromosomal companions. Chromosomes 10 and 16 are known to contain tumor suppressor genes. Not surprisingly, chromosome 16 contains six down regulated genes, three of which are essential for the encoding of metallothioneins. Metallothioneins bind the transition metal Zn⁺². Prostatic fluid contains large concentrations of Zn⁺², the function of which is largely unknown.

TABLE 1
Clinical and Histologic Details of Radical Prostatectomy Specimens and
Frozen Section in 9 Men with Gleason Grade 4/5 Cancers

| | | | | Index | % Grade | % Grade | Lymph | Seminal | |
|---|---|---|---|---|---|---|---|---|---|
| | | | Prostate | Cancer | 4/5 | 4/5 in | Node | Vesicle | Vascular |
| Sample | Age | PSA | Weight | Volume | Cancer in | Frozen | Status | Invasion | Invasion |
| Number | (Yrs) | (ng/ml | (ms) | (cc) | RP | Section | (+/0) | (+/0) | (# of foci) |
| 1 | 63 | 77 | 48 | 37 | 90 | 80 | 0 | 0 | 3 |
| 2a | 55 | 33 | 51 | 121 | 10 | >90 | 0 | 0 | 0 |
| 3 | 54 | 14 | 44 | 12.3 | 50 | 80 | 0 | 0 | 0 |
| 4 | 58 | 6.4 | 39 | 9.0 | 80 | 70 | 0 | 0 | 5 |
| 5 | 54 | 13 | 46 | 49 | 100 | 100 | 0 | + | 2 |
| 6^{a} | 54 | 14 | 36 | 110 | 100 | 100 | 0 | 0 | 2 |
| 7 | 69 | 6.0 | 37 | 3.9 | 80 | 80 | 0 | 0 | 1 |
| 8 | 50 | 15.5 | 35 | 10.0 | 85 | 85 | 0 | + | 4 |
| 9^{b,c} | 64 | 76 | 47 | 3.4 | 80 | 80 | 0 | + | 2 |

^{a}Cancer located in transition zone; all others located in peripheral zone.
^{b}Samples 9 and 14 (see TABLE 2) are from the same prostate.
^{c}Large secondary cancer 2.4 cc, 70% grade 4/5.
Average age of the 9 men with cancer was 58 years.

TABLE 2
Clinical and Histologic Details of Radical Prostatectomy Specimens in 8 Men
with Benign Prostatic Hyperplasia (BPH)^{a}

| | | | | | | | Seminal | |
|---|---|---|---|---|---|---|---|---|
| | | | Prostate | Index | % Gleason | Lymph | Vesicle | Vascular |
| Sample | Age | PSA | Weight | Cancer | Grade | Node | Invasion | Invasion |
| Number | (Yrs) | (n ml) | (ms) | Volume (cc) | 4/5 Cancer | Status (+/0) | (+/0) | (No. of foci) |
| 10 | 56 | 74 | 64^{s)} | 5.4 | 35 | 0 | + | 3 |
| 11 | 57 | 5.0 | 65 | 0.3 | 20 | 0 | 0 | 0 |
| 12 | 57 | 5.8 | 62 | 20 | 50 | 0 | 0 | 1 |
| 13 | 64 | 7.1 | 52 | 26 | 55 | 0 | 0 | 0 |
| 14 | 64 | 76 | 47 | 3.4 | 80 | 0 | + | 2 |
| 15 | 56 | 76 | 46 | 01 | 0 | 0 | 0 | 0 |
| 16 | 69 | 8.9 | 60 | 22 | 40 | 0 | 0 | 0 |

| | | |
|---|---|---|
| 17 | 71 | Cystoprostatectomy for bladder cancer No prostate cancer Prostate weight = 26 grams |

^{a}In none of these men was there any cancer in the frozen section of the BPH nodules.
^{b}Samples 9 (see TABLE 1) and 14 are from the same prostate.
Average age of the 8 men with BPH was 62 years.

TABLE 3
22 Up Regulated Genes (of 5,150) Selected By p-value Difference of 0.0005
Between nine Gleason grade 4/5 and eight BPH Prostate Tissues

| | | Average | | |
|---|---|---|---|---|
| Rank | Probe Set | Fold Change | Description | Locus |
| 1 | X07732 | 34 | Hepsin | 19q11-q132 |
| 2 | M99487 | 7 | Prostate specific membrane antigen/PSMA* | 11p11.2 |
| 3 | AB000584 | 7 | PLAB/Prostate differentiation factor/TGF-β | 19p13.1-13.2 |
| 4 | M30894 | 5 | T-cell Receptor Ti rearranged γ-chain* | 7p15-p14 |
| 5 | X04325 | 5 | GJB1/gap Junction protein | Xq13.1 |
| 6 | U19251 | 5 | Neuronal apoptosis inhibitory protein | 5q13 1 |
| 7 | D82345 | 4 | TMSNB/NB thymosin β | Xq21.33-q22.3 |
| 8 | D28589 | 4 | Human mRNA KIAA00167, partial sequence | NA |
| 9 | U24577 | 4 | PLA2G7/LDL- phospholipase A2 | NA |
| 10 | M16938 | 3 | Homeobox c8 protein | 12q12-q13 |
| 11 | M93036 | 3 | Human carcinoma associated antigen GA733-2 | NA |
| 12 | X87176 | 3 | HSD17B4/17β-hydroxsteroid dehydrogenase IV | 5q2 |
| 13 | U30999 | 3 | ALCAM/Activated leucocyte cell adhesion molecule | 3q13.1-13.2 |
| 14 | HG1612- | 3 | MacMARCKS | 1p34 |
| | HT1612 | | | |
| 15 | X80692 | 2 | ERK3 (extracellular signal-regulated kinase) | 15 21 |
| 16 | U37689 | 2 | RNA polymerase 11 subunit (hsRPB8) | NA |
| 17 | X89986 | 2 | NBK apoptotic inducer protein | 22q13.31 |
| 18 | M77836 | 2 | PYCRI/Pyrroline 5-carboxlate reductase I | Chrom. 17 |
| 19 | D13370 | 2 | APEX nuclease | 14q11.2-q12 |
| 20 | U41315 | 2 | Ring zinc-finer protein (ANF127-xp) | 15q11-q13 |
| 21 | J03592 | 2 | SLC25A6/Solute carrier family 25, member A6 | Xp22 32 or Yp |
| 22 | M83751 | 2 | Arginine-rich protein | 3p21 1 |

Note: These genes are ordered by their Fold Changes (FC) of 2 or greater in comparing the transcript expression levels between Gleason grade 4/5 cancer and BPH. *Genes known to be related to prostate cancer.

TABLE 4
64 Down Regulated Genes

| | | Average | | |
|---|---|---|---|---|
| Rank | Probe Set | Fold Change | Description | Locus |
| 1 | U35735 | 12 | SLC14A1 - urea transporter | 18q11-q12 |
| 2 | D00408 | 11 | CYP3A7 - cytochrome P-450 (P-450 HFLa) | 7q21-q22 1 |
| 3 | M21389 | 10 | KRT5 - keratin type II | 12 13 |
| 4 | U04313 | 10 | P15- protease inhibitor 5 (maspin) | 18q21.3 |
| 5 | L24203 | 10 | ATDC - ataxia-telangiectasia group D-associated | 11q22-q23 |
| | | | Protein | |
| 6 | J00124 | 9 | KRT14 (keratin 14)* | 17q12-q21 |
| 7 | X14885 | 9 | TGFB3 (transforming growth factor, beta 3) | 14q24 |
| 8 | J05459 | 8 | GSTM3 (glutathione transferase M3) | 1p13 3 |
| 9 | J03910 | 7 | MTIG (metallothionein 1G)* | 16q13 |
| 10 | L39833 | 6 | hKvBeta3 (potassium voltage-gated channel, beta | 3q26 1 |
| | | | member 3) | |
| 11 | U45955 | 6 | GPM6B (glycoprotein M6B) | Xp22 2 |
| 12 | U61374 | 5 | SRPX (sushi-repeat-containing protein, X | Xp211 |
| | | | Chromosome) | |
| 13 | M97639 | 5 | ROR2 (receptor tyrosine kinase-like orphan receptor | 9q22 |
| | | | 2) | |
| 14 | U48959 | 5 | MYLK (myosin, light polypeptide kinase)* | 3cen-q21 |
| 15 | X00129 | 5 | RBP (retinal binding protein) | 10q23-q24 |
| 16 | M32053 | 5 | H 19 RNA gene | 11p15.5 |
| 17 | D42108 | 5 | PLCE (phospholipase C, epsilon) | 2q33 |
| 18 | M62402 | 5 | IGFBP6 (insulin-like growth factor binding protein | 12g13 |
| | | | 6* | |
| 19 | D10667 | 4 | MYHI1 (myosin, heavy polypeptide 11, smooth | 16p13.13- |
| | | | muscle) | p13 12 |
| 20 | D88422 | 4 | CSTA (cystatin A or stefin A) | 3q21 |
| 21 | M63391 | 4 | DES (desmin)* | 2q35 |
| 22 | D83018 | 4 | NELL2 (nel (chicken)-like 2) | 12q13 11- |
| | | | | q13 12 |
| 23 | Z18951 | 4 | CAVI (caveolin 1, caveolae protein, 22kD)* | 7q31.1 |
| 24 | U28368 | 4 | ID4 (inhibitor of DNA binding 4, dominant negative | 6p22-p21 |
| | | | Helix-loop-helix protein) | |
| 25 | X66945 | 4 | FGFRI (fibroblast growth factor receptor 1) | 8p11.2-p11.1 |
| 26 | M57399 | 4 | HBNF-1 (nerve growth factor)* | 7q33 |
| 27 | U25138 | 4 | KCNMBI (potassium large conductance calcium- | 5q34 |
| | | | activated channel, subfamily M, beta member 1) | |
| 28 | U50360 | 4 | CAMK2G (calcium/calmodulin-dependent protein | 10q22 |
| | | | kinase (CaM kinase) II gamma) | |
| 29 | S45630 | 4 | CRYAB (crystalline, alpha B)* | 11q223- |
| | | | | q23.1 |
| 30 | HG3543-HT3 | 4 | IGFII (insulin-like growth factor 2)* | 11p155 |
| 31 | X89066 | 3 | TRPC1 (transient receptor potential channel 1) | 3q22-q24 |
| 32 | M24485 | 3 | GSTP1 (glutathione S-transferase pi)* | 11q13 |
| 33 | M63379 | 3 | TRPM-2 (testosterone-repressed prostate message | 8p21-p12 |
| | | | 2)* | |
| 34 | X78992 | 3 | BRF2 (butyrate response factor 2 (EGF-response | 2p22.3-p21 |
| | | | factor 2)) | |
| 35 | U77594 | 3 | RARRES2 (retinoic acid receptor responder | 7 |
| | | | (tazarotene induced) 2) | |
| 36 | L13698 | 3 | gas 1 gene | 9q21 3-22 1 |
| 37 | X52947 | 3 | GJA1 (gap junction protein, alpha 1, 43kD (connexin | 6q21-q23.2 |
| | | | 43))* | |
| | | | SERPINF1 (serine (or cysteine) proteinase inhibitor, | 17p13.1 |
| 38 | U29953 | 3 | clade F (alpha-2 antiplasmin, pigment epithelium | |
| | | | derived factor), member 1) | |
| 39 | U32114 | 3 | caveolin-2 | 7q31 1 |
| 40 | X96381 | 3 | ETV5 (ets variant gene 5 (ets-related molecule)) | 3q28 |
| 41 | D13628 | 3 | KIAA0003/ANGPT1 (angiopoietin 1) | 8q22.3-q23 |
| 42 | X76717 | 3 | MTIL (metallothionein IL) | 16q13 |
| 43 | D13639 | 3 | KIAK0002/CCND2 (cyclin D2) | 12p13 |
| 44 | M60828 | 3 | FGF7 (fibroblast growth factor 7 (keratinocyte | 15q15-q21.1 |
| | | | growth factor )* | |
| 45 | M33308 | 3 | VCL (vinculin) | 10q22 1-q23 |
| 46 | M83186 | 3 | COX7A1 (cytochrome c oxidase subunit VIIa | 19q13.1 |
| | | | of e tide 1 (muscle)) | |
| 47 | M14636 | 3 | PYGL (phosphorylase, glycogen; liver (Hers disease, | 14q21-q22 |
| | | | 1 co en rage disease type VI)) | |
| 48 | M55531 | 3 | SLC2A5 (solute carrier family 2 (facilitated glucose | Lp36 2 |
| | | | transporter), member 5) | |
| 49 | Y00097 | 3 | annexin VI (p68) | 5q32-q34 |
| 50 | L42176 | 3 | DRAL (downregulated in rhabdomyosarcoma lim | 2q12-q14 |
| | | | protein) | |
| 51 | D78014 | 3 | DPYSL3 (dihydropyrimidinase-like 3) | 5q32 |
| 52 | U95740_ma | 3 | A-362G6.1 (hypothetical protein A-362G6.1) | 16p13.1 |
| 53 | D45917 | 2 | TIMP3 (tissue inhibitor of metalloproteinase 3) | 22q12 3 |
| 54 | Z24725 | 2 | MIG2 (mitogen inducible 2) | 14 |
| 55 | U19495 | 2 | SDFI (stromal cell-derived factor 1) | 10q11 1 |
| 56 | D14695 | 2 | KIAA0025 (KIAA0025 gene product, MMS- | 16q12.2-q13 |
| | | | inducible gene) | |
| 57 | X83416 | 2 | PRNP (prion protein (p27-30)) | 2pter-p12 |
| 58 | L11005 | 2 | AOX1 (aldehyde oxidase 1) F | 2q33 |
| 59 | L09604 | 2 | PLP2 (proteolipid protein 2 (colonic epithelium- | Xp11 23 |
| | | | Enriched | |
| 60 | V00594 | 2 | MT2A (metallothioncin 2A) | 16q13 |
| 61 | M14949 | 2 | RRAS (related RAS viral (r-ras) oncogene homolog) | 19q13.3-qter |
| 62 | D00017 | 2 | LIP2 (lipocortin II) | 15q21-q22 |
| 63 | HG3432-HT3 | 2 | Fibroblast Growth Factor Receptor K-Sam* | 10q26 |
| 64 | U51336 | 2 | ITPKI (inositol 1,3,4-triphosphate 5/6 kinase)* | 14q31 |

Note: These genes are ordered by their Fold Changes (FC) of 2 or greater in comparing the transcript expression levels between BPH and Gleason grade 4/5 cancer. *Genes known to be related to prostate cancer.

TABLE 5
Chromosomal Location of 18 Up Regulated and 63 Down Regulated Genes

| Chromo- | | | | | | | | | | | | | | | | | | | | | | | | |
|---|---|---|---|---|---|---|---|---|---|---|---|---|---|---|---|---|---|---|---|---|---|---|---|---|
| some | 1 | 2 | 3 | 4 | 5 | 6 | 7 | 8 | 9 | 10 | 11 | 12 | 13 | 14 | 15 | 16 | 17 | 18 | 19 | 20 | 21 | 22 | X | Y |
| Up | 1 | | 2 | | 2 | | 1 | | | | 1 | 1 | | 1 | 2 | | 1 | | 2 | | | 1 | 3 | |
| Regulated | | | | | | | | | | | | | | | | | | | | | | | | |
| Down | 2 | 5 | 5 | | 3 | 2 | 5 | 3 | 2 | 5 | 4 | 4 | | 4 | 2 | 6 | 2 | 2 | 2 | 1 | | 1 | 3 | |
| Regulated | | | | | | | | | | | | | | | | | | | | | | | | |

1. A method for predicting the outcome of cancer in a patient, comprising the steps of:
comparing level of expression of at least one RNA transcript or its translation product from a first or a second group of RNA transcripts in a first sample of prostate tissue to level of expression of the transcripts or translation products in a second sample of prostate tissue wherein the first prostate tissue sample is neoplastic and the second prostate tissue sample is a nonmalignant human prostate tissue, wherein the first group of RNA transcripts consists of transcripts of genes selected from the group consisting of SLC14A1-urea transporter (U35735), CYP3AI-cytochrome P-450 (P-450 HFLa) (D00408), KRT5—keratin type II (M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione transferase M3) (J05459), hKvBeta3 (potassium voltage-gated channel, beta member 3) (L39833), GPM6B (glycoprotein M6B) (U45955), SRPX (sushi-repeat containing protein, X chromosome) (U61374), ROR2 (receptor tyrosine kinase-like orphan receptor 2) (M97639), RBP (retinol binding protein) (X00129), H19 RNA gene (M32053), PLCE (phospholipase C, epsilon) (D42108), MYHI 1 (myosin, heavy polypeptide II, smooth muscle) (D10667), CSTA (cystatin A (stefin A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMBI (potassium large conductance calcium-activated channel, subfamily M, beta member 1) (U25138), CAMK2G (calcium/calmodulin-dependent protein kinase (CaM kinase) II gamma) (U50360), TRPC 1 (transient receptor potential channel 1) (X89066), BRF2 (butyrate response factor 2 (EGF-response factor 2)) (X78992), RARRES2 (retinoic acid receptor responder (tazarotene induced) 2) (U77594), gasl gene (L13698), SERPINFI (serene or cysteine) proteinase inhibitor, clade F (alpha-2 antiplasmin, pigment epithelium derived factor, member 1) (U29953), caveolm-2 (U32114), ETV5 (ets variant gene 5 (ets-related molecule)) (X96381), KIAA0003/ANGPTI (angiopoietm 1) (D13628), MT1L (metallothionein 1L) (X76717), KIAK0002/CCND2 (cyclin D2) (D13639), VCL (vinculin) (M33308), COX7A1 (cytochrome c oxidase subunit Vila polypeptide 1 (muscle)) (M83186), PYGL (phosphorylase, glycogen; liver (Hers disease, glycogen storage disease type VI)) (M14636), SLC2A5 (solute carrier family 2 (facilitated glucose transporter), member 5) (M55531), annexin VI (p68) (Y00097), DRAL (L42176), DPYSL3 (dibydropyrimidinaselike 3) (D78014), A-362G6.1 (hypothetical protein A-362G6.1) (U95740_rna), TIMP3 (tissue inhibitor of metalloproteinase 3) (D45917), MIG2 (mitogen inducible 2 (Z24725), SDF1 (stromal cell-derived factor 1) (U19495), KIAA0025 (KIM0025 gene product; MMS-inducible gene) (D14695), PRNP (prion protein (p27-30)) (X83416), AOX1 (aldehyde oxidase 1) (L11005), PLP2 (proteolipid protein 2 (colonic epithelium-enriched)) (L09604), MT2A (metallothionein 2A) (V00594), RRAS (related RAS viral (r-ras) oncogene homolog) (M14949), and LIP2 (D00017) and wherein the second group of RNA transcripts consists of transcripts of genes selected from the group consisting of Hepsin (X07732), PLAB/Prostate differentiation factor/TGF-P (AB000584), GJB1/gap junction protein (X04325), Neuronal apoptosis inhibitory protein (U19251), TMSNB/NB thymosin β (D82345), Human mRNA KIM00167, partial sequence (D28589), PLA2G7/LDL-phospholipase A2 (U24577), Homeo box c8 protein (M16938), Human carcinoma associated antigen GA733-2 (M93036), HSD17B4/17β-hydroxysteroid dehydrogenase IV (X87176), ALCAM/Activated leucocyte cell adhesion molecule (U30999), Macmarcks (HG1612-HT1612), ERK3 (extracellular signal-regulated kinase) (X80692), RNA polymerase II subunit (hsRPB8) (U37689), NBK apoptotic inducer protein (X89986), PYCR1/Pyrroline 5-carboxylate reductase 1 (M77836), APEX nuclease (D13370), Ring zinc-forger protein (ANF127-xp) (U41315), SLC25A6/Solute carrier family 25, member A6 (J03592), and Arginine-rich protein (M83751);
identifying the patient as having a poor outcome when expression of at least one of the first group of RNA transcripts or translation products is found to be lower in the first sample than in the second sample, and expression of at least one of the second group of transcripts or translation products is found to be higher in the first sample than in the second sample.

2. The method of claim 1 further comprising the step of comparing transcripts or translation products of at least two of the genes of the first group is performed.

3. The method of claim 1 further comprising the step of comparing transcripts or translation products of at least two of the genes of the second group is performed.

4. The method of claim 1 further comprising the step of comparing transcripts or translation products of at least five of the genes of the first group is performed.

5. The method of claim 1 further comprising the step of comparing transcripts or translation products of at least five of the genes of the second group is performed.

6. The method of claim 1 further comprising the step of comparing transcripts or translation products of at least ten of the genes of the first group is performed.

7. The method of claim 1 further comprising the step of comparing transcripts or translation products of at least ten of the genes of the second group is performed.

8. The method of claim 1 further comprising the step of comparing transcripts or translation products of at least twenty of the genes of the first group is performed.

9. The method of claim 1 further comprising the step of comparing transcripts or translation products of at least twenty of the genes of the second group is performed.

10. The method of claim 1 further comprising the step of comparing transcripts or translation products of at least thirty of the genes of the first group is performed.

11. The method of claim 1 further comprising the step of comparing transcripts or translation products of at least forty-nine of the genes of the first group is performed.

12. The method of claim 1 wherein the at least one RNA transcript or its translation product of the first group comprises the transcript of the gene maspin (U04313).

13. The method of claim 1 wherein the at least one RNA transcript or its translation product of the second group comprises the transcript of the gene hepsin (X07732).\

14. The method of claim 1 further comprising the step of categorizing the patient as having a poor outcome when expression of the at least one RNA transcript or translation product of the first group is found to be at least 1.5 fold lower in the first tissue sample relative to the second tissue sample.

15. The method of claim 1 further comprising the step of comparing a transcript or translation product of the first group, said transcript or translation product being of a gene selected from the group consisting of SLC14A1-urea transporter (U35735), CYP3A1-cytochrome P-450 (P-450 HFLa) (D00408), KRT5—keratin type 11(M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione transferase M3) (J05459), hKvBeta3 (potassium voltage-gated channel, beta member 3) (L39833), GPM6B (glycoprotein M6B) (U45955), SRPX (sushi-repeat-containing protein, X chromosome) (U61374), ROR2 (receptor tyrosine kinase-like orphan receptor 2) (M97639), RBP (retinol binding protein) (X00129), H19 RNA gene (M32053), PLCE (phospholipase C, epsilon) (D42108), MYH11 (myosin, heavy polypeptide II, smooth muscle) (D10667), CSTA (cystatin A (stefin A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMB 1 (potassium large conductance calcium-activated channel, subfamily M, beta member 1) (U25138), and CAMK2G (calcium/calmodulin-dependent protein kinase (CaM kinase) II gamma) (U50360).

16. The method of claim 15 further comprising the step of identifying the patient as having a poor outcome when expression of the at least one RNA transcript or translation product of the first group is found to be at least 4 fold lower in the first tissue sample relative to the second tissue sample.

17. The method of claim 1 further comprising the step of identifying the patient as having a poor outcome when expression of the at least one RNA transcript or translation product of the second group is found to be at least 1.5 fold higher in the first tissue sample relative to the second tissue sample.

18. The method of claim 1 further comprising the step of comparing transcript or translation product of the second group, said transcript or translation product being of a gene selected from the group consisting of Hepsin (X07732), PLAB/Prostate differentiation factor/TGF-β (AB000584), GJB1/gap junction protein (X04325), Neuronal apoptosis inhibitory protein (U19251), TMSNB/NB thymosin β (D82345), Human mRNA KIAA00167, partial sequence (D28589), and PLA2G7/LDL-phospholipase A2 (U24577).

19. The method of claim 18 further comprising the step of categorizing the patient as having a poor outcome when expression of the at least one RNA transcript or translation product of the second group is found to be at least four fold higher in the first tissue sample relative to the second tissue sample.

20. The method of claim 1 further comprising the step of comparing the level of expression of at least one RNA transcript of the fast group in the first sample to the level of expression of said transcript in the second sample.

21. The method of claim 1 further comprising the step of determining the level of expression of RNA transcripts using an array of nucleic acid molecules.

22. The method of claim 1 further comprising the step of comparing the level of expression of at least one RNA transcript of the second group in the first sample to the level of expression of said transcript in the second sample.

23. The method of claim 1 further comprising the step of comparing transcripts or translation products of at least two genes in each of said first and said second groups.

24. The method of claim 1 further comprising the step of comparing transcripts or translation products of at least five genes in each of said first and said second groups.

25. The method of claim 1 further comprising the step of comparing transcripts or translation products of at least ten genes in each of said first and said second groups.

26. The method of claim 1 further comprising the step of comparing transcripts or translation products of at least twenty genes in each of said first and said second groups.

27. The method of claim 1 further comprising the step of comparing at least thirty transcripts or translation products in the first group and twenty transcripts or translation products in the second groups.

28. The method of claim 1 further comprising the step of comparing at least forty transcripts or translation products in the first group and twenty transcripts or translation products in the second group.

29. The method of claim 1 further comprising the step of comparing at least forty-nine transcripts or translation products in the first group and twenty transcripts or translation products in the second group.

30. The method of claim 1 further comprising the step of identifying the patient as having a poor outcome when expression of at least one RNA transcript or translation product of the first group is found to be at least 1.5-fold lower in the first sample relative to the second sample, and the expression of at least one of RNA transcript or translation product of the second group is found to be at least 1.5-fold higher in the first sample relative to the second sample.

31. The method of claim 1 further comprising the step of selecting said transcript or translation product of the first group from the group consisting of SLC14A1-urea transporter (U35735), QYP3A1-cytochrome P-450 (P-450 HFLa) (D00408), KRT5-keratin type II (M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione transferase M3) (J05459), hKvBeta3 (potassium voltage-gated channel, beta member 3) (L39833), GPM6B (glycoprotein M6B) (U45955), SRPX (sushi-repeat-containing protein, X chromosome) (U61374), ROR2 (receptor tyrosine kinase-like orphan receptor 2) (M97639), RBP (retinol binding protein) (X00129), H19 RNA gene (M32053), PLCE (phospholipase C, epsilon) (D42108), MYH11 (myosin, heavy polypeptide II, smooth muscle) (D10667), CSTA (cystatin A (stefin A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMB1 (potassium large conductance calcium-activated channel, subfamily M, beta member 1) (U25138), and CAMK2G (calcium/calmodulm-dependent protein kinase (CaM kinase) II gamma) (U50360) and selecting said transcript or translation product of the second group from the group consisting of Hepsin (X07732), PLAB/Prostate differentiation factor/TGF-β (AB000584), GJB1/gap junction protein (X04325), Neuronal apoptosis inhibitory protein (U19251), TMSNB/NB thymosin p (D82345), Human mRNA KIAA00167, partial sequence (D28589), and PLA2G7/LDL-phospholipase A2 (U24577).

32. The method of claim 31 further comprising the step of identifying the patient as having a poor outcome when expression of at least one of the first group of RNA transcripts or translation products is found to be at least 4.0-fold lower in the first sample relative to the second sample, and expression of at least one of the second group of RNA transcripts or translation products is found to be at least 4.0-fold higher in the first sample relative to the second sample.

33. The method of claim 1 wherein the neoplastic tissue comprises Gleason grade 4/5 prostate carcinoma cells.

34. The method of claim 1 wherein the nonmalignant human prostate tissue is benign prostate hyperplasia.

35. A method for evaluating carcinogenicity of an agent to human prostate cells comprising the steps of:
comparing level of expression of at least one transcript or its translation product from a first or a second group of RNA transcripts in a first sample of human prostate cells contacted with a test agent to level of expression in a second sample of human prostate cells not contacted with the test agent, wherein the first group of RNA transcripts consists of transcripts of genes selected from the group consisting of SLC14A1-urea transporter (U35735), CYP3A1-cytochrome P-450 (P-450 HFLa) (D00408), KRT5—keratin type II (M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione transferase M3) (J05459), hKvBeta3 (potassium voltage-gated channel, beta member 3) (L39833), GPM6B (glycoprotein M6B) (U45955), SRPX (sushi-repeat containing protein, X chromosome) (U61374), ROR2 (receptor tyrosine knase-like orphan receptor 2) (M97639), RBP (retinol binding protein) (X00129), H19 RNA gene (M32053), PLCE (phospholipase C, epsilon) (D42108), MYH11 (myosin, heavy polypeptide II, smooth muscle) (D10667), CSTA (cystatin A (stefin A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMBI (potassium large conductance calcium-activated channel, subfamily M, beta member 1) (U25138), CAMK2G (calcium/calmodulin-dependent protein kinase (CaM knase) II gamma) (U50360), TRPC 1 (transient receptor potential channel 1) (X89066), BRF2 (butyrate response factor 2 (EGF-response factor 2)) (X78992), RARRES2 (retinoic acid receptor responder (tazarotene induced) 2) (U77594), gasl gene (L13698), SERPINF1 (serine (or cysteine) proteinase inhibitor, clade F (alpha-2 antiplasmin, pigment epithelium derived factor), member 1) (U29953), caveolin-2 (U32114), ETV5 (ets variant gene 5 (ets-related molecule)) (X96381), KIAA0003/ANGPT1 (angiopoietin 1) (D13628), MT1L (metallothionein 1 L) (X76717), KIAK0002/CCND2 (cyclin 132) (D13639), VCL (vinculin) (M33308), COX7Al (cytochrome c oxidase subunit VIIa polypeptide 1 (muscle)) (M83186), PYGL (phosphorylase, glycogen; liver (Hers disease, glycogen storage disease type VI)) (M14636), SLC2A5 (solute carrier family 2 (facilitated glucose transporter), member 5) (M55531), annexin VI (p68) (Y00097), DRAL (L42176), DPYSL3 (dihydropyrimidinaselike 3) (D78014), A-362G6.1 (hypothetical protein A-362G6.1) (U95740 ma), TIMP3 (tissue inhibitor of metalloproteinase 3) (D45917), MIG2 (mitogen inducible 2 (Z24725), SDF1 (stromal cell-derived factor 1) (U19495), KIM0025 (KIM0025 gene product; MMS-inducible gene) (D14695), PRNP (prion protein (p27-30)) (X83416), AOX1 (aldehyde oxidase 1) (L 1005), PLP2 (proteolipid protein 2 (colonic epithelium-enriched)) (L09604), MT2A (metallothionein 2A) (V00594), RRAS (related RAS viral (r-ras) oncogene homolog) (M 14949), and LIP2 (D00017), and the second group of RNA transcripts consists of transcripts of genes selected from the group consisting of Hepsin (X07732), PLAB/Prostate differentiation factor/TGF-β (AB000584), GJB1/gap junction protein (X04325), Neuronal apoptosis inhibitory protein (U19251), TMSNB/NB thymosin β (D82345), Human mRNA KIAA00167, partial sequence (D28589), PLA2G7/LDL-phospholipase A2 (U24577), Homeo box c8 protein (M16938), Human carcinoma associated antigen GA733-2 (M93036), HSD17B4/17β-hydroxysteroid dehydrogenase IV (X87176), ALCAM/Activated leucocyte cell adhesion molecule (U30999), Macmarcks (HG1612-HT1612), ERK3 (extracellular signal-regulated kinase) (X80692), RNA polymerase II subunit (hsRPB8) (U37689), NBK apoptotic inducer protein (X89986), PYCR1/Pyrroline 5-carboxylate reductase 1 (M77836), APEX nuclease (D13370), Ring zinc-forger protein (ANF127-xp) (U41315), SLC25A6/Solute carrier family 25, member A6 (J03592), and Arginine-rich protein (M83751), wherein an agent which decreases the level of expression of at least one of the genes of the first group, or an agent which increases the level of expression of at least one of the genes in the second group is a potential carcinogen to human prostate cells.

36. The method of claim 35 further comprising the step of comparing the level of expression of at least two of the transcripts or translation products.

37. The method of claim 35 further comprising the step of comparing the level of expression of at least five of the transcripts or translation products.

38. The method of claim 35 further comprising the step of comparing the level of expression of at least ten of the transcripts or translation products.

39. The method of claim 35 further comprising the step of comparing the level of expression of at least twenty of the transcripts or translation products.

40. The method of claim 35 further comprising the step of comparing the level of expression of at least fifty of the transcripts or translation products.

41. The method of claim 35 further comprising the step of comparing the level of expression of at least sixty of the transcripts or translation products.

42. The method of claim 35 further comprising the step of comparing the level of expression of sixty-nine of the transcripts or translation products.

43. The method of claim 35 further comprising the step of determining the level of expression of RNA transcripts using an array of nucleic acid molecules.

44. The method of claim 35 further comprising the step of comparing the level of expression of at least one transcript.

45. A method of slowing progression of prostate cancer in a patient comprising the step of:
administering to prostate cancer cells of the patient a polynucleotide comprising a coding sequence of a gene selected from the group consisting of SLC14A1-urea transporter (U35735), CYP3A1-cytochrome P-450 (P-450 HFLa) (D00408), KRT5-keratin type II (M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione transferase M3) (J05459), hKvBeta3 (potassium voltage-gated channel, beta member 3) (L39833), GPM6B (glycoprotein M6B) (U45955), SRPX (sushi-repeat-containing protein, X chromosome) (U61374), ROR2 (receptor tyrosine kinase-like orphan receptor 2) (M97639), RBP (retinol binding protein) (X00129), H19 RNA gene (M32053), PLCE (phospholipase C, epsilon) (D42108), MYH11 (myosin, heavy polypeptide II, smooth muscle) (D10667), CSTA (cystatin A (stefin A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMB1 (potassium large conductance calcium-activated channel, subfamily M, beta member 1) (U25138), CAMK2G (calcium/calmodulm-dependent protein kmase (CaM kinase) II gamma) (U50360), TRPC1 (transient receptor potential channel 1) (X89066), BRF2 (butyrate response factor 2 (EGF-response factor 2)) (X78992), RARRES2 (retinoic acid receptor responder (tazarotene induced) 2) (U77594), gasl gene (L13698), SERPINF1 (serine (or cysteine) proteinase inhibitor, clade F (alpha-2 antiplasmin, pigment epithelium derived factor), member 1) (U29953), caveolin-2 (U32114), ETV5 (ets variant gene 5 (ets-related molecule)) (X96381), KIAA0003/ANGPT1 (angiopoietin 1) (D13628), MT1L (metallothionein 1 L) (X76717), KIAK0002/CCND2 (cyclin D2) (D13639), VCL (vinculin) (M33308), COX7A1 (cytochrome c oxidase subunit VIIa polypeptide 1 (muscle)) (M83186), PYGL (phosphorylase, glycogen; liver (Hers disease, glycogen storage disease type VI)) (M14636), SLC2A5 (solute carrier family 2 (facilitated glucose transporter), member 5) (M55531), annexin VI (p68) (Y00097), DRAL (L42176), DPYSL3 (dihydropyrimidinaselike 3) (D78014), A-362G6.1 (hypothetical protein A-362G6.1) (U95740_rna), TIMP3 (tissue inhibitor of metalloproteinase 3) (D45917), MIG2 (mitogen inducible 2 (Z24725), SDF1 (stromal cell-derived factor 1) (U19495), KIAA0025 (KIM0025 gene product; MMS-inducible gene) (D14695), PRNP (prion protein (p27-30)) (X83416), AOX1 (aldehyde oxidase 1) (L11005), PLP2 (proteolipid protein 2 (colonic epithelium-enriched)) (L09604), MT2A (metallothionein 2A) (V00594), RRAS (related RAS viral (r-ras) oncogene homolog) (M14949), and LIP2 (D00017), whereby the gene is expressed in the prostate cancer cells, thereby slowing progression of prostate cancer in the patient.

46. The method of claim 45 wherein said gene is maspin (U04313).

47. A method of slowing progression of prostate cancer in a patient, comprising the step of:
administering to prostate cancer cells of a patient an antisense construct comprising at least 12 nucleotides of a coding sequence of a gene selected from the group consisting of Hepsin (X07732), PLAB/Prostate differentiation factor/TGF-β (AB000584), GJB1/gap junction protein (X04325), Neuronal apoptosis inhibitory protein (U19251), TMSNB/NB thymosin β (D82345), Human mRNA KIAA00167, partial sequence (D28589), PLA2G7/LDL-phospholipase A2 (U24577), Homeo box c8 protein (M16938), Human carcinoma associated antigen GA733-2 (M93036), HSD17B4/17β-hydroxysteroid dehydrogenase IV (X87176), ALCAM/Activated leucocyte cell adhesion molecule (U30999), Macmarcks (HG1612-HT1612), ERK3 (extracellular signal-regulated kinase) (X80692), RNA polymerase II subunit (hsRPB8) (U37689), NBK apoptosic inducer protein (X89986), PYCR1/Pyrroline 5-carboxylate reductase 1 (M77836), APEX nuclease (D13370), Ring zinc-finger protein (ANF 127-xp) (U41315), SLC25A6/Solute carrier family 25, member A6 (J03592), and Arginine-rich protein (M83751), wherein the coding sequence is in a 3′ to 5′ orientation with respect to a promoter which controls its expression, whereby an antisense RNA is expressed in cells of the cancer, thereby slowing progression of prostate cancer in the patient.

48. The method of claim 47 wherein said gene is hepsin (X07732).

49. A method of slowing progression of prostate cancer in a patient, comprising the step of:
administering to prostate cancer cells of a patient an antibody which specifically binds to a protein expressed from a gene selected from the group consisting of Hepsin (X07732), PLAB/Prostate differentiation factor/TGF-β (AB000584), GJB1/gap junction protein (X04325), Neuronal apoptosis inhibitory protein (U19251), TMSNB/NB thymosin p (D82345), Human mRNA KIAA00167, partial sequence (D28589), PLA2G7/LDL-phospholipase A2 (U24577), Homeo box c8 protein (M16938), Human carcinoma associated antigen GA733-2 (M93036), HSD17B4/17β-hydroxysteroid dehydrogenase IV (X87176), ALCAM/Activated leucocyte cell adhesion molecule (U30999), Macmarcks (HG1612-HT1612), ERK3 (extracellular signal-regulated kinase) (X80692), RNA polymerase II subunit (hsRPB8) (U37689), NBK apoptotic inducer protein (X89986), PYCR1/Pyrroline 5-carboxylate reductase 1 (M77836), APEX nuclease (D13370), Ring zinc-finger protein (ANF127-xp) (U41315), SLC25A6/Solute carrier family 25, member A6 (J03592), and Arginine-rich protein (M83751), whereby the antibody binds to the protein, thereby slowing progression of prostate cancer in the patient.

50. The method of claim 49 wherein the antibody specifically binds hepsin.

51. A method of screening for candidate drugs useful in the treatment of prostate cancer, comprising the steps of:
contacting a prostate cancer cell with a test substance;
monitoring expression of a transcript or its translation product, wherein the transcript is of a gene selected from a first or a second group wherein the first group of genes is selected from the group consisting of SLC14A1-urea transporter (U35735), CYP3A1-cytochrome P-450 (P-450 HFLa) (D00408), KRT5—keratin type II (M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione transferase M3) (J05459), hKvBeta3 (potassium voltage-gated channel, beta member 3) (L39833), GPM6B (glycoprotein M6B) (U45955), SRPX (sushi-repeat-containing protein, X chromosome) (U61374), ROR2 (receptor tyrosine kinase-like orphan receptor 2) (M97639), RBP (retinol binding protein) (X00129), H19 RNA gene (M32053), PLCE (phospholipase C, epsilon) (D42108), MYH11 (myosin, heavy polypeptide II, smooth muscle) (D10667), CSTA (cystatin A (stefin A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMB1 (potassium large conductance calcium-activated channel, subfamily M, beta member 1) (U25138), CAMK2G (calcium/calmodulin-dependent protein kinase (CaM kinase) II gamma) (U50360), TRPC1 (transient receptor potential channel 1) (X89066), BRF2 (butyrate response factor 2 (EGF-response factor 2)) (X78992), RARRES2 (retinoic acid receptor responder (tazarotene induced) 2) (U77594), gas1 gene (L13698), SERPINF1 (serine (or cysteine) proteinase inhibitor, clade F (alpha-2 antiplasmin, pigment epithelium derived factor), member 1) (U29953), caveolin-2 (U32114), ETV5 (ets variant gene 5 (ets-related molecule)) (X96381), KIAA0003/ANGPT1 (angiopoietin 1) (D13628), MT1L (metallothionein 1L) (X76717), KIAK0002/CCND2 (cyclin D2) (D13639), VCL (vinculin) (M33308), COX7A1 (cytochrome c oxidase subumt VIIa polypeptide 1 (muscle)) (M83186), PYGL (phosphorylase, glycogen; liver (Hers disease, glycogen storage disease type VI)) (M14636), SLC2A5 (solute carrier family 2 (facilitated glucose transporter), member 5) (M55531), annexin VI (p68) (Y00097), DRAL (L42176), DPYSL3 (dihydropyrimidinase-like 3) (D78014), A-362G6.1 (hypothetical protein A-362G6.1) (U95740 ma), TIMP3 (tissue inhibitor of metalloproteinase 3) (D45917), MIG2 (mitogen inducible 2 (Z24725), SDF1 (stromal cell-derived factor 1) (U19495), KIAA0025 (KIAA0025 gene product; MMS-inducible gene) (D14695), PRNP (prion protein (p27-30)) (X83416), AOX1 (aldehyde oxidase 1) (L11005), PLP2 (proteolipid protein 2 (colonic epithelium-enriched)) (L09604), MT2A (metallothionein 2A) (V00594), RRAS (related RAS viral (r-ras) oncogene homolog) (M14949), and LIP2 (D00017), and the second group is selected from the group consisting of Hepsin (X07732), PLAB/Prostate differentiation factor/TGF-β (AB000584), GJB1/gap junction protein (X04325), Neuronal apoptosis inhibitory protein (U19251), TMSNB/NB thymosin p (D82345), Human mRNA KIAA00167, partial sequence (D28589), PLA2G7/LDL-phospholipase A2 (U24577), Homeo box c8 protein (M16938), Human carcinoma associated antigen GA733-2 (M93036), HSD17B4/17β-hydroxysteroid dehydrogenase IV (X87176), ALCAM/Activated leucocyte cell adhesion molecule (U30999), Macmarcks (HG1612-HT1612), ERK3 (extracellular signal-regulated kinase) (X80692), RNA polymerase II subunit (hsRPB8) (U37689), NBK apoptosic inducer protein (X89986), PYCR1/Pyrroline 5-carboxylate reductase 1 (M77836), APEX nuclease (D13370), Ring zinc-finger protein (ANF127-xp) (U41315), SLC25A6/Solute carrier family 25, member A6 (J03592), and Arginine-rich protein (M83751); and
identifying a test substance as a candidate drug useful for treating prostate cancer if it increases expression of at least one of the genes in the first group or decreases expression of at least one of the genes in the second group.

52. The method of claim 51 further comprising the step of identifying the test substance as a candidate drug if it increases expression of at least two of the genes selected from the first group or decreases expression of at least two of the genes selected from the second group.

53. The method of claim 51 further comprising the step of identifying the test substance as a candidate drug if it increases expression of at least five of the genes selected from the first group or decreases expression of at least five of the genes selected from the second group.

54. The method of claim 51 further comprising the step of identifying the test substance as a candidate drug if it increases expression of at least ten of the genes selected from the first group or decreases expression of at least ten of the genes from the second group.

55. The method of claim 51 further comprising the step of identifying the test substance as a candidate drug if it increases expression of at least twenty of the genes selected from the first group or decreases expression of at least twenty of the genes selected from the second group.

56. The method of claim 51 further comprising the step of determining the level of expression of RNA transcripts using an array of nucleic acid molecules.

57. The method of claim 51 further comprising the step of monitoring expression of at least one transcript.

58. A method for diagnosing prostate cancer in a patient, comprising the steps of:
comparing level of expression of at least one RNA transcript or its translation product in a test sample of prostate tissue to level of expression of the at least one transcript or translation product in a control sample of prostate tissue, wherein the test sample of prostate tissue is suspected of being neoplastic and the control sample is nonmalignant prostate tissue, wherein the at least one RNA transcript or its translation product is selected from a first or a second group of RNA transcripts or translation products, wherein the first group of RNA transcripts consists of transcripts of genes selected from the group consisting of SLC14A1-urea transporter (U35735), CYP3A1-cytochrome P-450 (P-450 HFLa) (D00408), KRT5—keratin type II (M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione transferase M3) (J05459), hKvBeta3 (potassium voltage-gated channel, beta member 3) (L39833), GPM6B (glycoprotein M6B) (U45955), SRPX (sushi-repeat-containing protein, X chromosome) (U61374), ROR2 (receptor tyrosine kinase-like orphan receptor 2) (M97639), RBP (retinol binding protein) (X00129), H19 RNA gene (M32053), PLCE (phospholipase C, epsilon) (D42108), MYH11 (myosin, heavy polypeptide II, smooth muscle) (D10667), CSTA (cystatin A (stefin A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMB 1 (potassium large conductance calcium-activated channel, subfamily M, beta member 1) (U25138), CAMK2G (calcium/calmodulin-dependent protein kinase (CaM kinase) II gamma) (U50360), TRPC1 (transient receptor potential channel 1) (X89066), BRF2 (butyrate response factor 2 (EGF-response factor 2)) (X78992), RARRES2 (retinoic acid receptor responder (tazarotene induced) 2) (U77594), gas1 gene (L13698), SERPINF1 (serine (or cysteine) proteinase inhibitor, clade F (alpha-2 antiplasmin, pigment epithelium derived factor), member 1) (U29953), caveolin-2 (U32114), ETV5 (ets variant gene 5 (ets-related molecule)) (X96381), KIAA0003/ANGPT1 (angiopoietin 1) (D13628), MT1L (metallothionein 1L) (X76717), KIAK0002/CCND2 (cyclin D2) (D13639), VCL (vinculin) (M33308), COX7Al (cytochrome c oxidase subunit VIIa polypeptide 1 (muscle)) (M83186), PYGL (phosphorylase, glycogen; liver (Hers disease, glycogen storage disease type VI)) (M14636), SLC2A5 (solute carrier family 2 (facilitated glucose transporter), member 5) (M55531), annexin VI (p68) (Y00097), DRAL (L42176), DPYSL3 (dihydropyrimidinase-like 3) (D78014), A-362G6.1 (hypothetical protein A-362G6.1) (U95740_rna), TIMP3 (tissue inhibitor of metalloproteinase 3) (D45917), MIG2 (mitogen inducible 2 (Z24725), SDF1 (stromal cell-derived factor 1) (U19495), KIAA0025 (KIM0025 gene product; MMS-inducible gene) (D14695), PRNP (prion protein (p27-30)) (X83416), AOX1 (aldehyde oxidase 1) (LI 1005), PLP2 (proteolipid protein 2 (colonic epithelium-enriched)) (L09604), MT2A (metallothionein 2A) (V00594), RRAS (related RAS viral (r-ras) oncogene homolog) (M14949), and LIP2 (D00017), and wherein the second group of RNA transcripts consists of transcripts of genes selected from the group consisting of Hepsin (X07732), PLAB/Prostate differentiation factor/TGF-β (AB000584), GJB1/gap junction protein (X04325), Neuronal apoptosis inhibitory protein (U19251), TMSNB/NB thymosin β (D82345), Human mRNA KIAA00167, partial sequence (D28589), PLA2G7/LDL-phospholipase A2 (U24577), Homeo box c8 protein (M16938), Human carcinoma associated antigen GA733-2 (M93036), HSD17B4/17β-hydroxysteroid dehydrogenase IV (X87176), ALCAM/Activated leucocyte cell adhesion molecule (U30999), Macmarcks (HG1612-HT1612), ERK3 (extracellular signal-regulated kinase) (X80692), RNA polymerase II subunit (hsRPB8) (U37689), NBK apoptosic inducer protein (X89986), PYCR1/Pyrroline 5-carboxylate reductase 1 (M77836), APEX nuclease (D13370), Ring zinc-finger protein (ANF127-xp) (U41315), SLC25A6/Solute carrier family 25, member A6 (J03592), and Arginine-rich protein (M83751); and
identifying the test sample as cancerous when expression of at least one of the first group of RNA transcripts or translation products is found to be lower in the test sample than in the control sample, and expression of at least one of the second group of transcripts or translation products is found to be higher in the test sample than in the control sample.

59. The method of claim 58 further comprising the step of determining the level of expression of RNA transcripts using an array of nucleic acid molecules.

60. The method of claim 58 further comprising the step of comparing the level of expression of at least one RNA transcript in the test sample to the level of expression of said transcript in the control sample.

61. The method of claim 58 further comprising the step of comparing transcripts or translation products of at least two of the genes of the first group.

62. The method of claim 58 further comprising the step of comparing transcripts or translation products of at least two of the genes of the second group.

63. The method of claim 58 further comprising the step of comparing transcripts or translation products of at least five of the genes of the first group.

64. The method of claim 58 further comprising the step of comparing transcripts or translation products of at least five of the genes of the second group.

65. The method of claim 58 further comprising the step of comparing transcripts or translation products of at least ten of the genes of the first group.

66. The method of claim 58 further comprising the step of comparing transcripts or translation products of at least ten of the genes of the second group.

67. The method of claim 58 further comprising the step of comparing transcripts or translation products of at least twenty of the genes of the first group.

68. The method of claim 58 further comprising the step of comparing transcripts or translation products of at least twenty of the genes of the second group.

69. The method of claim 58 further comprising the step of comparing transcripts or translation products of at least thirty of the genes of the first group.

70. The method of claim 58 further comprising the step of comparing transcripts or translation products of at least forty-nine of the genes of the first group.

71. The method of claim 58 further comprising the step of determining the expression level of maspin (U04313) transcript or its translation product.

72. The method of claim 58 further comprising the step of determining the expression level of hepsin (X07732) transcript or its translation product.

73. The method of claim 58 further comprising the step of comparing transcripts or translation products of at least two of the genes in each group of RNA transcripts or translation products.

74. The method of claim 58 further comprising the step of comparing transcripts or translation products of at least five of the genes in each group of transcripts or translation products.

75. The method of claim 58 further comprising the step of comparing transcripts or translation products of at least ten of the genes in each group of transcripts or translation products.

76. The method of claim 58 further comprising the step of comparing transcripts or translation products of at least twenty of the genes in each group of transcripts or translation products.

77. The method of claim 58 further comprising the step of comparing at least thirty of the transcripts or translation products in the fast group and twenty of the transcripts or translation products in the second group.

78. The method of claim 58 further comprising the step of comparing at least forty of the transcripts or translation products in the first group and twenty of the transcripts or translation products in the second group.

79. The method of claim 58 further comprising the step of comparing at least forty-nine of the transcripts or translation products in the first group and twenty of the transcripts or translation products in the second group.

80. The method of claim 58 wherein the at least one RNA transcript or its translation product of the first group of RNA transcripts or translation products comprises the transcript of the gene maspin (U04313).

81. The method of claim 58 wherein the at least one RNA transcript or its translation product of the second group of RNA transcripts comprises the transcript of the gene hepsin (X07732).

82. The method of claim 58 wherein the test sample comprises Gleason grade 4/5 prostate carcinoma cells.

83. The method of claim 58 wherein the nonmalignant prostate tissue is benign prostate hyperplasia tissue.

84. The method of claim 58 further comprising the step of identifying the test sample as Gleason grade 4/5 prostate carcinoma.

85. An array of nucleic acid molecules in which the nucleic acid molecules comprise a set of members having distinct sequences, wherein each member is fixed at a distinct location on the array, wherein at least 10% of the members on the array comprise at least 15 contiguous nucleotides of genes selected from the group consisting of SLC14A1-urea transporter (U35735), CYP3A1-cytochrome P-450 (P-450 HFLa) (D00408), KRT5—keratin type II (M21389), P15—protease inhibitor 5 (maspin) (U04313), ATDC—ataxia-telangiectasia group D-associated protein (L24203), TGFB3 (transforming growth factor, beta 3) (X14885), GSTM3 (glutathione; transferase M3) (J05459), hKvBeta3 (potassium voltage-gated channel, beta member 3) (L39833), GPM6B (glycoprotein M6B) (U45955), SRPX (sushi-repeatcontaining protein, X chromosome) (U61374), ROR2 (receptor tyrosine kinase-like orphan receptor 2) (M97639), RBP (retinol binding protein) (X00129), H19 RNA gene (M32053), PLCE (phospholipase C, epsilon) (D42108), MYH11 (myosin, heavy polypeptide II, smooth muscle) (D10667), CSTA (cystatin A (stein A)) (D88422), NELL2 (nel (chicken)-like 2) (D83018), ID4 (inhibitor of DNA binding 4, dominant negative helix-loop-helix protein) (U28368), FGFR1 (fibroblast growth factor receptor 1) (X66945), KCNMB1 (potassium large conductance calcium-activated channel, subfamily M, beta member 1) (U25138), CAMK2G (calcium/calmodulin-dependent protein kinase (CaM kinase) II gamma) (U50360), TRPC1 (transient receptor potential channel 1) (X89066), BRF2 (butyrate response factor 2 (EGF-response factor 2)) (X78992), RARRES2 (retinoic acid receptor responder (tazarotene induced) 2) (U77594), gas1 gene (L13698), SERPINF1 (serine (or cysteme) proteinase inhibitor, clade F (alpha-2 antiplasmin, pigment epithelium derived factor), member 1) (U29953), caveolin-2 (U32114), ETV5 (ets variant gene 5 (ets-related molecule)) (X96381), KIAA0003/ANGPT1 (angiopoietin 1) (D13628), MT1L (metallothionein 1L) (X76717), KIAK0002/CCND2 (cyclin D2) (D13639), VCL (vinculin) (M33308), COX7A1 (cytochrome c oxidase subunit VIIa polypeptide 1 (muscle)) (M83186), PYGL (phosphorylase, glycogen; liver (Hers disease, glycogen storage disease type VI)) (M14636), SLC2A5 (solute carrier family 2 (facilitated glucose transporter), member 5) (M55531), annexin VI (p68) (Y00097), DRAL (L42176), DPYSL3 (dihydropyrimidinaselike 3) (D78014), A-362G6.1 (hypothetical protein A-362G6.1) (U95740_rna), TIMP3 (tissue inhibitor of metalloproteinase 3) (D45917), MIG2 (mitogen inducible 2 (Z24725), SDF1 (stromal cell-derived factor 1) (U19495), KIM0025 (KIM0025 gene product; MMS-inducible gene) (D14695), PRNP (prion protein (p27-30)) (X83416), AOX1 (aldehyde oxidase 1) (L11005), PLP2 (proteolipid protein 2 (colonic epithelium-enriched)) (L09604), MT2A (metallothionem 2A) (V00594), RRAS (related RAS viral (r-ras) oncogene homolog) (M14949), LIP2 (D00017), Hepsin (X07732), PLAB/Prostate differentiation factor/TGF-β (AB000584), GJBI/gap junction protein (X04325), Neuronal apoptosis inhibitory protein (U19251), TMSNB/NB thymosin β (D82345), Human mRNA KIAA00167, partial sequence (D28589), PLA2G7/LDL-phospholipase A2 (U24577), Homeo box c8 protein (M16938), Human carcinoma associated antigen GA733-2 (M93036), HSD17B4/17β-hydroxysteroid dehydrogenase IV (X87176), ALCAM/Activated leucocyte cell adhesion molecule (U30999), Macmarcks (HG1612-HT1612), ERK3 (extracellular signal-regulated kmase) (X80692), RNA polymerase II subunit (hsRPB8) (U37689), NBK apoptosic inducer protein (X89986), PYCR1/Pyrroline 5-carboxylate reductase I (M77836), APEX nuclease (D13370), Ring zinc-finger protein (ANF127-xp) (U41315), SLC25A6/Solute carrier family 25, member A6 (J03592), and Arginine-rich protein (M83751).

86. The array of claim 85 wherein at least 20% of the members are selected from the group.

87. The array of claim 85 wherein at least 30% of the members are selected from the group.

88. The array of claim 85 wherein at least 40% of the members are selected from the group.

89. The array of claim 85 wherein at least 50% of the members are selected from the group.

90. A method for monitoring prostate cancer in a patient, comprising:
measuring level of at least one serum marker in a serum sample of a patient having prostate cancer, wherein the serum marker is a protein expressed from a first or second group of genes, wherein the first group of genes consists of P15-protease inhibitor 5 (mapsin) (U04313), TGFB3 (transforming growth factor, beta 3) (X14885), IGFBP6 (insulinlike growth factor binding protein 6) (M62402), NELL2 (nel (chicken)-like 2) (D83018), nerve growth factor (HBNF-1) (M57399), insulin-like growth factor 2 (HG3543-HT3739), SERPINF1 (serine (or cysteine) proteinase inhibitor, clade F (alpha-2 antiplasmin, pigment epithelium derived factor), member 1) (U29953), KIAA0003/ANGPT1 (angiopoietin 1) (D13628), FGF7 (fibroblast growth factor 7), TIMP (tissue inhibitor of metalloproteinase 3) (D45917), and SDF1 (stromal cell-derived factor 1) (U19495) and the second group of genes consists of the gene PLA2G7/LDL-phospholipase A2 (U24577).

91. The method of claim 90 further comprising the step of identifying the patient as having a poor outcome when level of the serum marker from the first group is found to be reduced in the patient relative to serum of an individual with a nonmalignant prostate or when level of the serum marker of the second group is found to be increased in the patient relative to serum of an individual with a nonmalignant prostate.

92. The method of claim 90 wherein the at least one serum marker measured is a protein expressed from a gene selected from the group consisting of P15-protease inhibitor 5 (mapsin) (U04313), TGFB3 (transforming growth factor, beta 3) (X14885), IGFBP6 (insulinlike growth factor binding protein 6) (M62402), NELL2 (nel (chicken)-like 2) (D83018), nerve growth factor (HBNF-1) (M57399), and insulin-like growth factor 2 (HG3543HT3739).

93. The method of claim 90 wherein the prostate cancer comprises Gleason grade 4/5 prostate carcinoma cells.

94. The method of claim 91 wherein the nonmalignant prostate comprises benign prostate hyperplasia.

95. The method of claim 90 further comprising the step of measuring the level of expression of at the least one serum marker by an antibody.

96. A method of diagnosing prostate cancer in a patient comprising:
measuring level of at least one serum marker in serum of a patient suspected of having prostate cancer, wherein the serum marker is a protein expressed from a first or second group of genes, wherein the first group of genes is selected from the group consisting of P15-protease inhibitor 5 (mapsin) (U04313), TGFB3 (transforming growth factor, beta 3) (X14885), IGFBP6 (insulin-like growth factor binding protein 6) (M62402), NELL2 (nel (chicken)-like 2) (D83018), nerve growth factor (HBNF-1) (M57399), insulin-like growth factor 2 (HG3543-HT3739), SERPINF1 (serine (or cysteme) proteinase inhibitor, clade F (alpha-2 antiplasmin, pigment epithelium derived factor), member 1) (U29953), KIAA0003/ANGPT 1 (angiopoietin 1) (D13628), FGF7 (fibroblast growth factor 7), TIMP (tissue inhibitor of metalloproteinase 3) (D45917), and SDF1 (stromal cell-derived factor 1) (U19495), and the second group of genes consists of the gene PLA2G7/LDL-phospholipase A2 (U24577);
identifying the patient as having prostate carcinoma when level of the serum marker from the first group is found to be reduced in the serum of the patient relative to an individual with a nonmalignant prostate or level of the serum marker from the second group is found to be increased in the serum of the patient relative to an individual with a nonmalignant prostate.

97. The method of claim 96 wherein the serum marker measured is a protein expressed from a gene of the first group selected from the group consisting of P15-protease inhibitor 5 (mapsin) (U04313), TGFB3 (transforming growth factor, beta 3) (X14885), IGFBP6 (insulinlike growth factor binding protein 6) (M62402), NELL2 (nel(chicken)-like 2) (D83018), nerve growth factor (HBNF-1) (M57399), and insulin-like growth factor 2 (HG3543-HT3739).

98. The method of claim 96 further comprising the step of identifying the prostate cancer as Gleason grade 4/5 prostate carcinoma.

99. The method of claim 96 wherein the nonmalignant prostate comprises benign prostate hyperplasia.

100. The method of claim 96 further comprising the step of measuring the level of expression of at the least one serum marker by an antibody.
